(19) United States
(12) Patent Application Publication
(10) Pub. No.: US 2024/0294462 A1
(43) Pub. Date: Sep. 5, 2024

(54) Method for the Synthesis of Ionizable Lipids Using a Doubly Alkylated Intermediate

(71) Applicant: NanoVation Therapeutics Inc., Vancouver (CA)

(72) Inventors: Fariba Saadati, Vancouver (CA); Huy Tran, Vancouver (CA); Marco Ciufolini, Vancouver (CA); N. D. Prasad Atmuri, Vancouver (CA)

(73) Assignee: NanoVation Therapeutics Inc., Vancouver (CA)

(21) Appl. No.: 18/442,431

(22) Filed: Feb. 15, 2024

Related U.S. Application Data
(60) Provisional application No. 63/445,854, filed on Feb. 15, 2023.

(51) Int. Cl. C07C 227/16; C07C 213/00; C07C 213/02; C07C 319/02

(52) U.S. Cl. CPC C07C 227/16; C07C 213/00; C07C 213/02; C07C 319/02

(57) ABSTRACT

Provided herein is a method for the preparation of ionizable, cationic amino lipids using a doubly alkylated nucleophilic intermediate to produce a ketone. The ketone, or a corresponding alcohol, is subjected to one or more synthesis steps to add an ionizable moiety thereto. The method can be advantageously employed for the synthesis of unsymmetrical analogues of the above lipids that would be considerably more difficult to make by alternative strategies. The method can also be used to prepare symmetrical ionizable, cationic amino lipids with fewer steps and/or with the use of fewer hazardous chemicals than known synthesis methods.

### INCORPORATION BY REFERENCE TO PRIORITY DOCUMENT

This application claims priority to U.S. Provisional Patent Application Serial No. 63/445,854, filed on Feb. 15, 2023, which is hereby expressly incorporated herein by reference in its entirety.

### TECHNICAL FIELD

Provided herein is a method for the preparation of ionizable lipids. The ionizable lipids may be formulated in a delivery vehicle so as to facilitate the encapsulation of a wide range of therapeutic agents or prodrugs therein, such as, without limitation, nucleic acids (e.g., RNA or DNA), proteins, peptides, pharmaceutical drugs and salts thereof.

### BACKGROUND

Therapeutic nucleic acids have enormous potential in medicine. To realize this potential, however, the nucleic acid should be delivered to a target site in a patient. This presents challenges since nucleic acid is rapidly degraded by enzymes in the plasma upon administration. Even if the nucleic acid is delivered to a disease site, there still remains the challenge of intracellular delivery. To address these problems, lipid nanoparticles (LNPs) have been developed that protect nucleic acid from such degradation and facilitate delivery across cellular membranes to gain access to the intracellular compartment, where the relevant translation machinery resides.

A key component of LNPs containing therapeutic nucleic acids is an ionizable lipid. The ionizable lipid is a non-naturally occurring substance that is typically positively charged at low pH, facilitating association with the negatively charged nucleic acid. However, the ionizable lipid is neutral at physiological pH, making it more biocompatible in biological systems. Further, it has been suggested that after the lipid nanoparticles are taken up by a cell by endocytosis, the ionizability of these lipids at the low pH of the endosome/lysosome enables endosomal escape, releasing the nucleic acid cargo into the intracellular compartment.

An ionizable lipid shown to exhibit in vivo efficacy for the delivery of nucleic acid therapeutics is D-Lin-KC2-DMA, 1 (Scheme 1), more simply referred-to as KC2 (Semple, S. S., et al., Nature Biotechnology 2010, 28, 172-U18; incorporated herein by reference). A later developed analogue of KC2 is D-Lin-MC3-DMA, 2 (Jayaraman, M., et al., Angew. Chem. Int. Ed. 2012, 51, 8529, incorporated herein by reference), referred to as MC3. MC3 is the ionizable component of Onpattro®, a lipid nanoparticle-based short interfering RNA (siRNA) drug for the treatment of polyneuropathies induced by hereditary transthyretin amyloidosis. The structures of KC2 and MC3 are provided below:

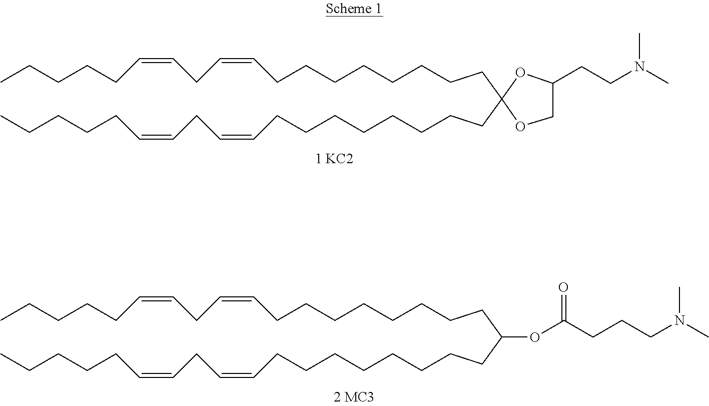

Onpattro® is the first siRNA-based medication to be approved by the Food and Drug Administration (FDA) and the European Medicines Evaluation Agency (EMEA). As a consequence, MC3 is considered a state-of-the art ionizable lipid for the delivery of nucleic acids. For example, in the case of siRNA, MC3 requires about three times less nucleic acid than KC2 in order to achieve the same biological response. However, KC2 is superior in other applications, and it remains a valuable research tool. Furthermore, it has recently been discovered that (i) certain formulations of therapeutic mRNA containing KC2 are considerably more efficacious than the best available alternatives (see PCT/CA2023/051451) and (ii) LNP formulations of plasmid DNA comprising KC2 are especially potent for the in vivo delivery of the nucleic acids (Algarni, A., et al., Biomater. Sci. 2022, 10, 2940, incorporated herein by reference).

The published synthesis of KC2 and MC3, comprises chemical steps that are best avoided in a pharmaceutical production plant, such as a Grignard reaction and an oxidation of an alcohol to a ketone with PCC, a hazardous reagent based on carcinogenic chromium(VI).

Accordingly, there is a need in the art for an improved synthesis of KC2, MC3, and related ionizable lipids, that avoids such problematic steps.

### Definitions

As used herein, the term “alkyl” or “alkyl group” is a C₁ to C₄₀ carbon-containing chain that is linear, cyclic (monocyclic or polycyclic) and/or branched and that optionally comprises C═C double bonds and/or one or more substituent ring structures, and that is optionally substituted.

As used herein, the term “Cₘ to Cₙ alkyl” or “Cₘ to Cₙ alkyl group” refers to a linear, cyclic and/or branched carbon chain having a total minimum of m carbon atoms and up to n carbon atoms, and that is optionally unsaturated and optionally substituted. For example, a “C₁ to C₃ alkyl” or “C₁ to C₃ alkyl group” is an alkyl having between 1 and 3 carbon atoms. The term “ring structure” is a 3- to 22-membered monocyclic or polycyclic alkyl

ring that is optionally substituted and optionally unsaturated. In some non-limiting examples, the ring structure is a 3- to 16-membered monocyclic or polycyclic alkyl ring that is optionally substituted. In further examples, the ring structure is a 3- to 8-membered monocyclic or polycyclic alkyl ring that is optionally substituted.

The term “monocyclic” is an optionally substituted alkyl group that is a single ring or that comprises a single ring substituent.

The term “polycyclic” is optionally substituted alkyl group that is, or comprises as a substituent(s), two or more ring structures that are chemically bonded to each other or two or more discrete ring structures.

The term “optionally substituted” with reference to an alkyl or alkyl group means that at least one hydrogen atom of the alkyl group can be replaced by a non-hydrogen atom or group of atoms (i.e., a “substituent”), and/or the alkyl is interrupted (i.e.., a -(CH)2- group replaced) by a heteroatom atom or one or more substituents, including but not limited to those comprising heteroatoms selected from O, S and NR′, wherein R′is as defined below. Non-limiting examples of atoms or substituents that may replace a hydrogen atom include halogen; deuterium; an alkyl group; a cycloalkyl group (mono or polycyclic); an oxo group (═O); a hydroxyl group (—OH); —(C═O)OR′; —O(C═O)R′; —C(═O)R′; O(C═O)OR′—; —OR′; —S(O)ₓR′; —SR′, —S—SR′; —C(═O)SR′; —SC(═O)R′; —NR′R′; —NR′C(═O)R′; —C(—O)NR′R′; —NR′C(═O)NR′R′; —OC(═O)NR′R′; —NR′C(═O)OR′; —NR′S(O)ₓNR′R′; —NR′S(O)ₓR′; and —S(O)ₓNR′R′, wherein R′ at each occurrence is independently selected from H, C₁-C₁₅ alkyl or cycloalkyl, and x is 0, 1 or 2. Non-limiting examples of atoms or substituents that may replace a carbon atom (interrupt the alkyl) include cycloalkyl groups (mono or polycyclic); —O—; —(C═O)O—; —O(C═O)—; —C(═O); —O(C═O)O—; —S(O)ₓ-; —S—; —S—S—; —C(═O)S—; —SC(═O)—; —NR′—; —NR′C(═O)—; —C(═O)NR′—; —NR′C(═O)NR′—; —OC(═O)NR′; —NR′C(═O)OR′—; —NR′S(O)ₓNR′—; —NR′S(O)ₓR′—; and —S(O)ₓNR′—, wherein R′ at each occurrence is independently selected from H, C₁-C₁₅ alkyl or cycloalkyl, and x is 0, 1 or 2.

As used herein, the term “ionizable lipid” refers to a lipid that, at a given pH, is in an electrostatically neutral form and that may either accept or donate protons, thereby becoming electrostatically charged, and for which the electrostatically neutral form has a calculated logarithm of the partition coefficient between water and 1-octanol (i.e., a cLogP) that is greater than 8.

The terms, “protonatable amino head group”, “ionizable head group” or “head group” are used interchangeably herein and refer to a moiety of the ionizable, cationic amino lipid that comprises the nitrogen atom in its head group that accepts a proton, thereby becoming electrostatically positively charged at a pH below its pKa. The protonatable amino head group has a central carbon atom to which each of the two lipophilic chains are directly bonded.

### SUMMARY

The present disclosure is based, at least in part, on the observation that certain ionizable lipids that are useful for the delivery of therapeutic agents, such as nucleic acids, are advantageously synthesized using mono- or dialkylation of chemical reagents such as 1-((isocyanomethyl)sulfonyl)-4-methylbenzene (tosylmethyl isocyanide; TosMIC), or methyl((methylsulfinyl)methyl) sulfane (Ogura reagent). In certain advantageous embodiments, the method described herein can avoid the use of more technically challenging and/or hazardous reactions and reagents and/or produces the desired lipids in fewer steps relative to known syntheses, thus potentially realizing considerable economies in terms of operator time and cost of materials.

According to one aspect of the disclosure, there is provided a method for producing an ionizable, cationic amino lipid, the method comprising:
(i) reacting a double alkylated nucleophilic intermediate under conditions effective to convert the intermediate to a ketone,
wherein the double alkylated nucleophilic intermediate has a structure as defined below:

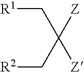

wherein Z is isocyano and Z′ is tosyl, or wherein Z is SCH₃ and Z′ is S(O)CH₃;
wherein the ketone has a structure as defined below:

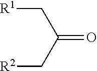

wherein the R¹ and R² groups are linear, branched and/or cyclic, optionally substituted C₃ to C₃₀ alkyl groups, optionally comprising 0-3 carbon-carbon double bonds, optionally comprising heteroatoms selected from N, O and/or S, optionally comprising homocyclic or heterocyclic ring structures; and
wherein R¹ and R² are identical or different; and
(ii) preparing the ionizable, cationic amino lipid from the ketone thereof using one or more synthesis steps resulting in an addition of an ionizable head group moiety to (a) the ketone; or (b) an alcohol produced from an optional reduction of the ketone to produce the alcohol, thereby producing the ionizable, cationic amino lipid, wherein the ionizable, cationic amino lipid has a protonatable amino head group and comprises two lipophilic chains comprising the R¹ and R² groups respectively and wherein the ionizable, cationic amino lipid has (i) a pKa of between 6 and 7.5; and (ii) a logP of at least 11.

According to one embodiment, the conditions effective to convert the double alkylated nucleophilic intermediate to the ketone comprise addition of a mineral acid.

According to one embodiment, the mineral acid is HCl, H₂SO₃, H₃PO₄ or a combination thereof.

According to a further embodiment, the double alkylated intermediate is produced by alkylating a reagent A selected from 1-((isocyanomethyl)sulfonyl)-4-methylbenzene (tosylmethyl isocyanide; TosMIC) or methyl((methylsulfinyl)methyl) sulfane (Ogura reagent) with at least one alkyl halide or sulfonate.

In another embodiment, R¹ and R² are identical, such that the ionizable, cationic amino lipid so produced is symmetrical, and wherein the double alkylated nucleophilic intermediate is produced upon treatment of the reagent A with a base and the alkyl halide or sulfonate.

According to a further embodiment, the double alkylated nucleophilic intermediate is produced using two or more molar equivalents each of the base and the alkyl halide or sulfonate.

In another embodiment, R¹ and R² are different, such that the ionizable, cationic amino lipid so produced is unsymmetrical, and wherein the ketone is produced by a reaction scheme as defined below comprising treating the reagent A with a first base and a first of the at least one alkyl halide or sulfonate having structure B to produce an alkylated intermediate of structure E, and reacting the structure E with a second base that is the same or different than the first base with a second of the at least one alkyl halide or sulfonate having structure F to produce the double alkylated nucleophilic intermediate G and wherein the conditions effective to produce the ketone H comprise reacting the double alkylated nucleophilic intermediate G with an aqueous solution of a strong mineral acid,

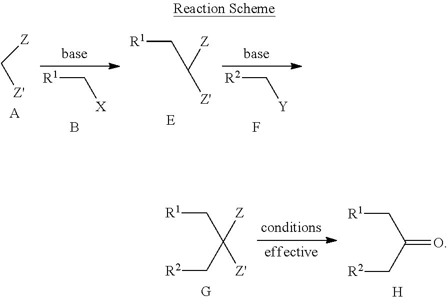

According to a further embodiment, the synthetic intermediate E is reacted with about one or more molar equivalent each of the second base and the structure F alkyl halide or sulfonate.

According to another embodiment, the reagent A is

methyl((methylsulfinyl)methyl)sulfane (Ogura reagent, Ogura sulfoxide) and the first and/or second base is potassium hydride.

In another embodiment, the reagent A is 1-((isocyanomethyl)sulfonyl)-4-methylbenzene (tosyl methyl isonitrile, TosMIC) and the first and/or second base is sodium hydride.

According to another embodiment, the ketone is converted into the corresponding alcohol by the reduction reaction comprising addition of the sodium borohydride in the solvent.

In another embodiment, the ketone is converted into the ionizable, cationic amino lipid by a ketalization with an aminodiol hydrochloride.

In a further embodiment, X and Y are selected from Cl, Br, I, tosyloxy and mesyloxy,

According to a further embodiment, the ketone is reacted, using one or more synthetic steps, to produce the ionizable, cationic amino lipid and wherein the ionizable, cationic amino lipid has a structure as defined by structure K:

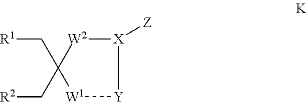

or a pharmaceutically acceptable salt thereof,
wherein:
   W¹ and Y are either bonded to each other or not bonded to each other (as indicated by the dashed bond), and:
   if W′ and Y are bonded to each other then:
      W¹ is O or S,
      W² is O or S,
      X is CH,
      Y is (CH₂)ₘ, wherein m is 1 or 2;
      Z is a group selected from structures a-c below:

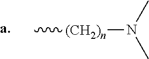

type 2 ionizable head;

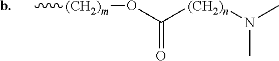

type 3 ionizable head; and

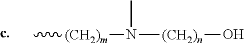

type 4 ionizable head,
   wherein the wavy line represents the bond to X,
   if W¹ and Y are not bonded to each other, then:
      W¹ is H;
      W² is O or S or NH or NR², wherein R² is a C₁ to C₄ small alkyl optionally substituted with an OH group;

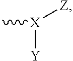

wherein the wavy line represents the bond to W², is a group chosen from among structures d-I below:

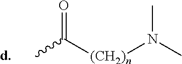

if W² is O, type 1 ionizable head;

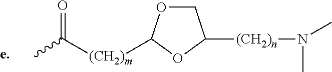

if W² is O, type 5 ionizable head;

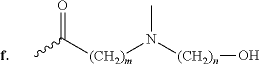

if W² is O, type 6 ionizable head;

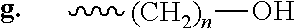

if W² is NH or NR², type 7 ionizable head;

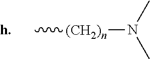

if W² is NH or NR², type 8 ionizable head; and

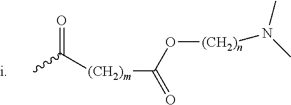

if W² is O, type 9 ionizable head, wherein the wavy line represents the bond to W². In one embodiment, one or more methylene group of any one of the foregoing ionizable head groups is substituted with S or O. In one embodiment, one or more methylene group of the foregoing Type 7 ionizable head groups is substituted with S or O.

In one embodiment, the ionizable, cationic amino lipid is MC3 or KC2.

In a further embodiment, the method further comprises formulating the ionizable, cationic amino lipid within a lipid nanoparticle.

In a further embodiment, the ionizable, cationic amino lipid is co-formulated with nucleic acid, non-cationic lipid and optionally a conjugated lipid that inhibits aggregation of particles.

According to another embodiment, the non-cationic lipid is phosphatidylcholine, cholesterol or a combination thereof and wherein the conjugated lipid is a hydrophilic polymer-lipid conjugate.

According to a further aspect, there is provided a use of a double alkylated nucleophilic intermediate to produce an ionizable, cationic amino lipid, the double alkylated nucleophilic intermediate having a structure as defined below:

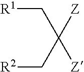

wherein Z is SCH₃ and Z′ is S(O)CH₃, or wherein Z is isocyano and Z′ is tosyl;

wherein the R¹ and R² groups are linear, branched and/or cyclic, optionally substituted C₃ to C₃₀ alkyl groups, optionally comprising 0-3 carbon-carbon double bonds, optionally comprising one or more heteroatoms selected from N, O and/or S, optionally comprising one or more homocyclic or heterocyclic ring structures; and wherein R¹ and R² are identical or different.

Other objects, features, and advantages of the present disclosure will be apparent to those of skill in the art from the following detailed description and figures.

### BRIEF DESCRIPTION OF THE DRAWINGS

FIG. 1 outlines the known synthesis of (6Z,9Z,28Z,31Z)-heptatriaconta-6,9,28,31-tetraen-19-ol (the precursor of MC3) and (6Z,9Z,28Z,31Z)-heptatriaconta-6,9,28,31-tetraen-19-one (the precursor of KC2).

FIG. 2 outlines the known conversion of (6Z,9Z,28Z,31Z)-heptatriaconta-6,9,28,31-tetraen-19-ol to MC3 and of (6Z,9Z,28Z,31Z)-heptatriaconta-6,9,28,31-tetraen-19-one to KC2.

FIG. 3 outlines the inventive synthesis of (6Z,9Z,28Z,31Z)-heptatriaconta-6,9,28,31-tetraen-19-one and of (6Z,9Z,28Z,31Z)-heptatriaconta-6,9,28,31-tetraen-19-ol according to the present disclosure, and their conversion to KC2 and MC3, respectively.

### DETAILED DESCRIPTION

Ionizable, cationic amino lipids such as KC2, MC3, and analogs thereof can be advantageously prepared from an appropriate ketone by chemical methods that transform the ketone group into an ionizable head group. Representative, but by no means limiting, examples of how said ketone group can be transformed into an ionizable head group are provided in co-pending and co-owned applications (WO 2022/246568, WO 2022/246571, WO 2023/147657, PCT/CA2023/051272, PCT/CA2023/051273, PCT/CA2023/051727, PCT/CA2023/051274, each incorporated herein by reference). Consequently, the synthesis of KC2, MC3, and analogs thereof can start with the assembly of a suitable ketone building block.

In some examples, the present disclosure is based on the finding that ketone building blocks used for the synthesis of ionizable lipids for the delivery of nucleic acid or other polyanionic therapeutics can be advantageously produced via a doubly alkylated nucleophilic intermediate as described herein. The chemical synthesis of the ionizable lipids is thus simplified. Specifically, the method herein may avoid technically challenging transformations such as Grignard reactions, and/or steps that require toxic/carcinogenic reagents, such as oxidations with Cr(VI) reagents. Furthermore, the ionizable lipids may be produced in fewer synthesis steps than known methods. Ionizable lipids that can thus be manufactured more safely, with fewer steps, and/or more economically include, without limitation, D-Lin-KC2-DMA and D-Lin-MC3-DMA, among others.

In some embodiments, the present disclosure is based on the discovery that a product of double alkylation of 1-((isocyanomethyl)sulfonyl)-4-methylbenzene (tosylmethyl isocyanide; TosMIC) or methyl((methylsulfinyl)methyl)sulfane (Ogura reagent) with an alkyl sulfonate or halide, such as those derived from a fatty alcohol (e.g., a sulfonate or halide derivative of linoleyl alcohol), can be converted into a ketone suitable for the preparation of ionizable lipids (e.g., without limitation, KC2 and MC3) under conditions that may preserve the C=C double bonds in the alkyl chains (e.g., avoids double bond isomerization). In addition, the chemical synthesis of ionizable lipids (including but not limited to KC2) by the present disclosure requires fewer steps and/or is more economical relative to known methods.

In some embodiments, the foregoing ketone is symmetrical, in the sense that the two alkyl groups bonded to the carbonyl (C═O) carbon are identical. Such a symmetrical ketone is shown as structure D in Synthetic Diagram A below:

The Z-CH₂-Z′ compound (structure A) is a reagent selected from 1-((isocyanomethyl)sulfonyl)-4-methylbenzene (more commonly known as tosyl methyl isonitrile, TosMIC) or methyl((methylsulfinyl)methyl)sulfane (more commonly known as the Ogura reagent or Ogura sulfoxide). In such embodiments, Z is isocyano and Z′ is tosyl Z (TosMIC), or Z is —SCH₃ and Z′ is S(O)CH₃ (Ogura reagent).

R¹ is a linear or branched, optionally substituted C₃ to C₃₀ alkyl group, optionally comprising 0-3 carbon-carbon double bond(s), optionally comprising one or more heteroatoms such as N, O and/or S, optionally comprising one or more homocyclic or heterocyclic ring structures.

X is a leaving group such as Cl, Br, I, or sulfonyloxy such as tosyloxy, mesyloxy, and the like; i.e., R¹—CH₂—X is an alkyl halide or an alkyl sulfonate.

The preparation of synthetic intermediate C may be advantageously carried out in a single synthetic step upon treatment of reagent A with two or more molar equivalents each of base and an alkyl halide or sulfonate of general structure B. Thus, compound C is directly obtained in the first step of the reaction sequence in Synthetic Scheme A.

Synthetic intermediate C is then subjected to “conditions effective” for its conversion to the ketone of structure D. For example, the conditions effective to produce structure D comprise a treatment of compound C with an aqueous solution of a strong mineral acid such as HCl, H2SO₃, H₃PO₄ and the like.

In some embodiments, it is advantageous to isolate synthetic intermediate C and subject it to the conditions effective to produce structure D in a distinct synthetic step. The preparation of ketone D may thus take place in two distinct synthetic steps.

In other embodiments, it is advantageous not to isolate synthetic intermediate C, but to subject it to the conditions effective to produce structure D in the same synthetic step that led to its formation. This can be done, for example, by adding an aqueous solution of a strong mineral acid such as HCl, H2SO₃, H₃PO₄ and the like, to the reaction mixture in which C was formed, or by transferring the reaction mixture in which C was formed into an aqueous solution of a strong mineral acid such as HCl, H₂SO₃, H₃PO₄ and the like. The preparation of ketone D may thus take place in a single synthetic step.

In other embodiments, the aforementioned ketone is unsymmetrical, in the sense that the two alkyl groups bonded to the carbonyl (C═O) carbon are different. Such an unsymmetrical ketone is shown as structure H in Synthetic Diagram B below, wherein:

Reagents Z—CH₂—Z′ (structure A) and R¹—CH₂—X (structure B) are as defined for Synthetic Diagram A above.

R² is a linear or branched, optionally substituted C₃ to C₃₀ alkyl group, optionally comprising 0-3 carbon-carbon double bond(s), optionally comprising one or more heteroatoms such as N, O and/or S, optionally comprising one or more homocyclic or heterocyclic ring structures, wherein R² is different from R^{1.}

Y is a leaving group such as Cl, Br, I, or sulfonyloxy such as tosyloxy, mesyloxy, and the like; i.e.., R²—CH₂—Y is an alkyl halide or an alkyl sulfonate.

The “conditions effective” for the conversion of compound G into unsymmetrical ketone H are as described above for Synthetic Diagram A.

For example, the inventors have determined that the Ogura reagent or TosMIC can be selectively mono-alkylated with an alkyl sulfonate or halide under suitable reaction conditions. The product of mono-alkylation thus obtained, compound E, can be alkylated a second time with a different alkyl sulfonate or halide of structure F to produce a doubly alkylated product of structure G, which can be converted into unsymmetrical ketone H.

Ketones such as D or H can be transformed into ionizable lipids that would be difficult and less economical to synthesize by alternative methods.

According to another example of the disclosure, the carbonyl group in a ketone of structure D in Synthetic Diagram A, or its derivative, or in a ketone of structure E in Synthetic Diagram B above, or its derivative, can be used to add an ionizable head group moiety to the structure, as described in co-owned and co-pending WO 2022/246555, WO 2022/246571; WO 2022/246568; WO 2023/147657, PCT/CA2023/051272; PCT/CA2023/051273; PCT/CA2023/051727 and PCT/CA2023/051274, each hereby incorporated by reference.

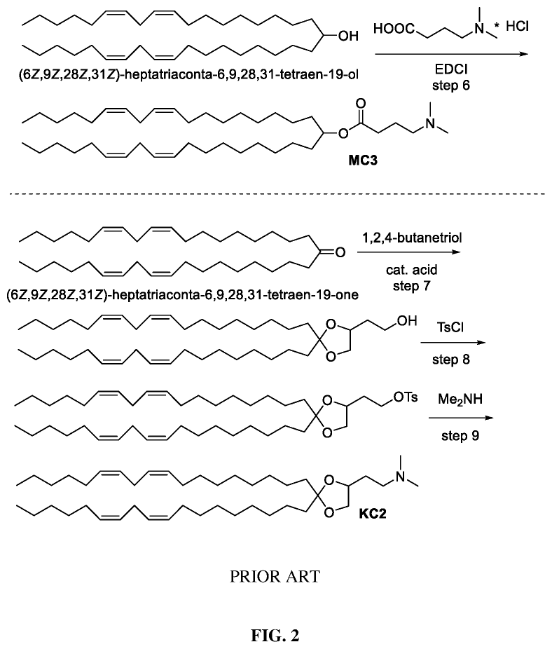

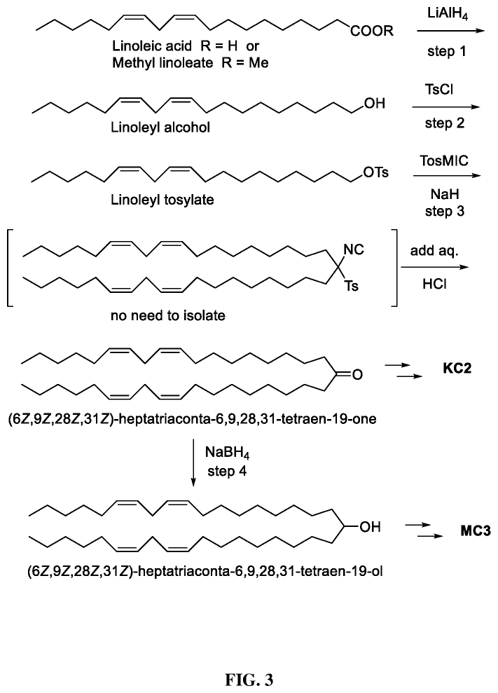

A non-limiting example of producing an ionizable lipid from a symmetrical ketone of structure D is shown in FIG. 2, which depicts the conversion of ketone, (6Z,9Z,28Z,31Z)-heptatriaconta-6,9,28,31-tetraen-19-one to the ionizable lipid, KC2. An example of a complete synthesis of KC2 from the ketone is described in Example 1 and FIG. 3 herein.

A non-limiting example of producing an ionizable lipid from an unsymmetrical ketone of structure H is shown in Example 2 herein.

In an alternative embodiment, an ionizable lipid may be prepared from an alcohol produced from an optional reduction of the ketone. The reagent for reducing the ketone to its corresponding alcohol may serve as a source of hydride that functions as a hydride nucleophile for the reduction. The addition of the hydride anion to the ketone produces an alkoxide anion, and a protonation results in the corresponding alcohol. An example of a reagent that can be used in the reduction step is sodium borohydride (NaBH4). Reagents such as lithium aluminum hydride (LAH, LiAlH₄), diisobutyl aluminum hydride (DIBAL, i-Bu₂AlH), triisobutyl aluminum (TIBA, i-Bu₃Al), aluminum isopropoxide [(i-PrO)₃Al], and related reagents, may be used as well if desired, although they are more hazardous and less convenient to use than NaBH₄. Furthermore, LAH, DIBAL and TIBA pose a fire hazard and react violently with water, alcohols and acidic groups. Thus, in one embodiment, the reduction of the ketone to a corresponding alcohol does not include the addition of LAH, DIBAL, and TIBA.

As would be appreciated by those of skill in the art, conversion of the ketone to the corresponding alcohol with the reducing agent is typically carried out in a suitable solvent. An alcohol solvent, such as methanol, ethanol, propanol, isopropanol, butanol, isobutanol, and the like, may be used in the reduction when the reduction is carried out with NaBH₄. Other suitable solvents known to those of skill in the art may be used when the reduction is carried out with reducing agents other than NaBH4.

A non-limiting example of an ionizable lipid produced from an alcohol is shown in FIG. 2, which depicts the conversion of (6Z,9Z,28Z,31Z)-heptatriaconta-6,9,28,31-tetraen-19-ol to MC3. An example synthesis of MC3 from the alcohol is described in Example 1 herein.

Because the ketone group in a compound such as D or H can be transformed into diverse ionizable head groups, as described in the above-referenced co-pending and co-owned applications, the person of skill in the art will recognize that the method of this disclosure allows for the production of a wide range of ionizable lipids. In other words, KC2 and MC3 are merely exemplary.

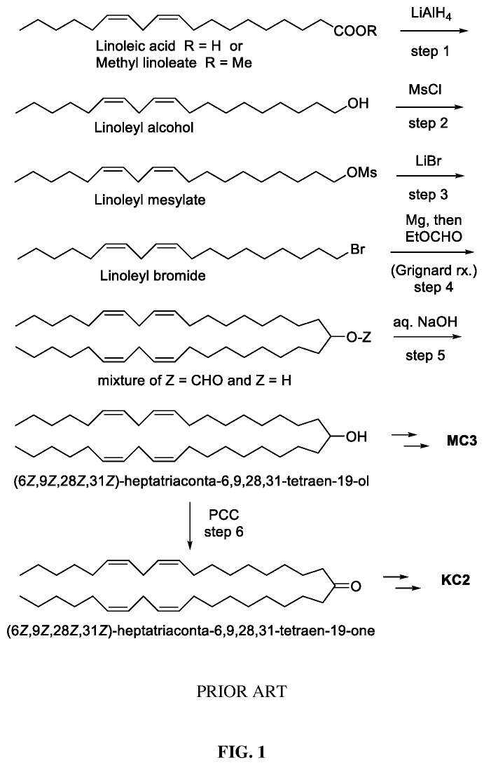

In order to specifically demonstrate the improved economics of the disclosed method, a comparison can be made between the example syntheses of the inventive method and known methods for producing KC2 and MC3 ionizable lipids. The synthesis of KC2 and MC3 using known methods starts with the preparation of alcohol 9 as shown in Scheme 2 below and FIG. 1. Accordingly, reduction of linoleic acid, 3, or a corresponding ester such as methyl linoleate, 4, with lithium aluminum hydride (LAH) produces 5, which is converted into bromide 7 by way of mesylate 6. A first problematic step of the synthesis is the formation of a Grignard reagent from 7, followed by in situ reaction with excess ethyl formate to give a mixture of desired alcohol 9 and its undesired formate ester, 8. The mixture of 8 and 9 is thus subjected in a separate step to treatment with aqueous sodium hydroxide to convert any 8 into 9. It should be noted that while Grignard reactions can be carried out in pharmaceutical plants, they are best avoided because of the hazardous and pyrophoric nature of Grignard reagents and their moisture sensitivity. This necessitates operation under rigorously anhydrous conditions and under inert atmosphere. Furthermore, appropriate safety measures must be taken during the

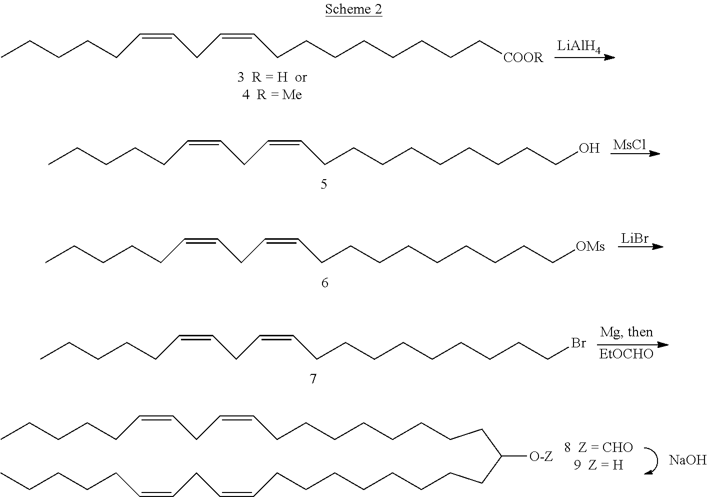

conduct of such reactions. Each of these requirements adds significantly to the cost of the synthesis. Alcohol 9 can be converted into MC3 in one step, by esterification of the OH group with 4-(dimethylamino)-butanoic acid hydrochloride in the presence of a condensing agent. Conversely, the synthesis of KC2 from 9 requires an additional four synthetic steps (Scheme 3), the first one of which, the oxidation of 9 to ketone 10, is problematic. This reaction can be carried out with pyridinium chlorochromate (PCC), which is based on toxic, carcinogenic Cr(VI). The use of Cr(VI) oxidants is best avoided in a pharmaceutical production plant, because stringent safety measures would have to be implemented to use Cr(VI) reagents and dispose of chromium-containing reaction byproducts. Furthermore, reaction products would have to be subject to rigorous analysis and purification to ascertain that no metal contamination is present, adding significantly to the cost of the synthesis process. An additional difficulty is that pyridinium chlorochromate (PCC) and related Cr(VI) reagents tend to attack the sensitive CH₂ located between the carbon-carbon double bonds, resulting in formation of byproducts. This further complicates the purification of the desired 10. Fortunately, the remaining steps of the synthesis are straightforward and pose no significant difficulties.

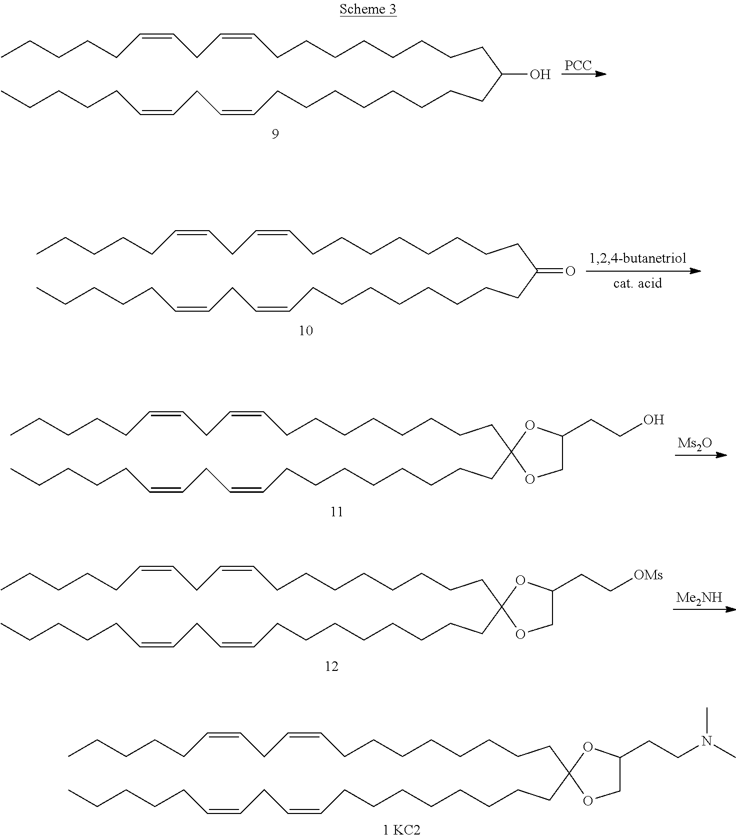

The inventors have recognized that a more economical and industrially viable synthesis of 1 avoids not only the Grignard reaction but also the oxidation step. Such an alternative synthesis of 1 produces ketone 10 directly.

To illustrate the improved economics of the present disclosure, the inventive synthesis of KC2 and MC3 according to non-limiting examples of the invention is set forth below. Such exemplary synthesis is based on a double alkylation of 1-((isocyanomethyl)sulfonyl)-4-methylbenzene (tosylmethyl isocyanide; TosMIC), or methyl((methylsulfinyl)methyl)sulfane (Ogura reagent), with a sulfonate or a halide derivative of alcohol 5. The reaction can be carried out in a solvent such as sulfolane, tetrahydrofuran (THF), dimethyl sulfoxide (DMSO), dimethylformamide (DMF), dimethylacetamide (DMA), N,N,-dimethyl propyleneurea (DMPU), at a temperature between −20° C. and +80° C., under inert atmosphere (N₂ or Ar), and in the presence of a hydride strong base such as NaH (e.g., for TosMIC) or KH (e.g., for the Ogura reagent), a metal alkoxide such as potassium tert-butoxide (t-BuOK), and the like.

Alternatively, reactions involving TosMIC can be carried out in a mixed organic-aqueous medium at a temperature between 0° C. and +100° C., under inert atmosphere (N₂ or Ar), and in the presence of a quaternary ammonium hydroxide such as tetrabutylammonium hydroxide, benzyl trimethylammonium hydroxide, and the like. An appropriate quantity of an alkali metal iodide (Lil, NaI, or KI) may be optionally added to the reaction mixture to accelerate the rate of the alkylation step.

Without intending to be limiting, this is exemplified in Scheme 4 with the synthesis of 10 from TosMIC, 14. Double alkylation of 14 produces 15, which can be isolated and fully characterized. However, in certain embodiments, it is expedient not to isolate it, but transfer the reaction solution in which 15 was formed into an aqueous solution of a strong mineral acid such as HCl, H₂SO₄ or H₃PO₄, or add an aqueous solution of a strong mineral acid such as HCl, H₂SO₄ or H₃PO₄ to the reaction mixture in which 15 was formed, resulting in direct conversion of 15 to ketone 10. Thus, ketone 10 can be made by the new route in a total of three steps from linoleic acid or an ester thereof (LAH reduction, conversion of the resulting alcohol into a sulfonate, and double alkylation/in situ hydrolysis), without using Grignard or oxidation steps.

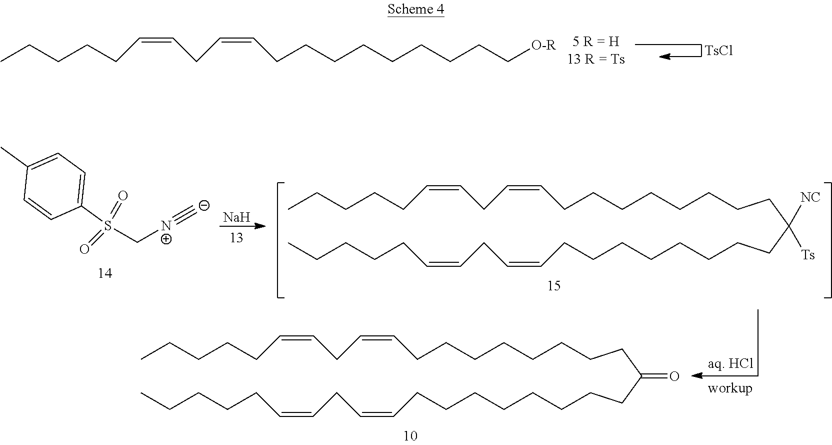

In another embodiment, the double alkylation of TosMIC can be achieved in an aqueous-organic medium by the use of a base such a tetraalkylammonium hydroxide instead of the more hazardous NaH. For example (Scheme 5), the reaction of TosMIC with a linoleyl halide, such as bromide, 7, or sulfonate, such as tosylate 13, in a mixture of water and THF,

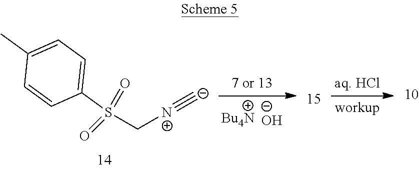

and in the presence of tetrabutylammonium hydroxide, at a temperature between 0 and 100° C., leads to 15, which can then be transformed into 10 as per Scheme 4 above.

The ketone can be further converted into KC2 in three synthetic steps, or into MC3 in two steps (Scheme 6). Thus, the inventive method makes KC2 available in a total of 6 steps, instead of 9, from linoleic acid or an ester thereof, and MC3 is now available in a total of 5 steps, instead of 6, also from linoleic acid or an ester thereof, substantially reducing the cost of the synthesis.

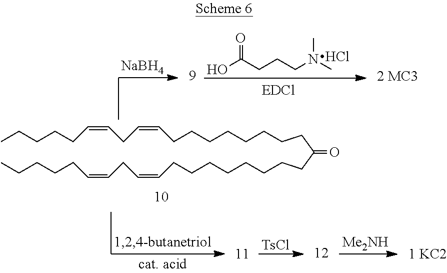

In another embodiment, the method of this disclosure allows the sequential alkylation of TosMIC with two different alkyl halides or sulfonates, leading to the formation of unsymmetrical ketones. Without intending to be limiting, the double alkylated intermediate can be used to prepare ketones such as 16-18 of Scheme 7. These ketones are available by alternative routes, and can be converted into the exemplary unsymmetrical lipids 33-35 of Scheme 12 below.

The synthesis of 16 can be achieved, for example, starting with the mono-alkylation of TosMIC with oleyl bromide, 19 or tosylate, 20 (Scheme 8). The product of this step, 21, is

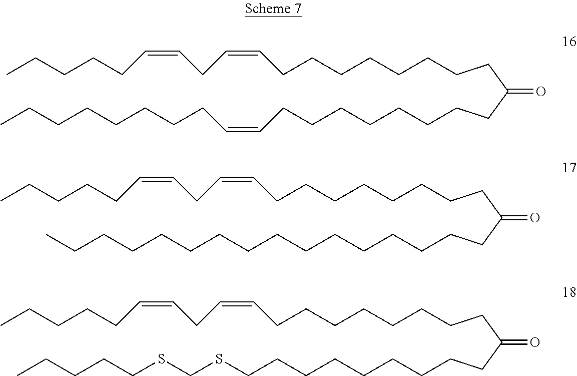

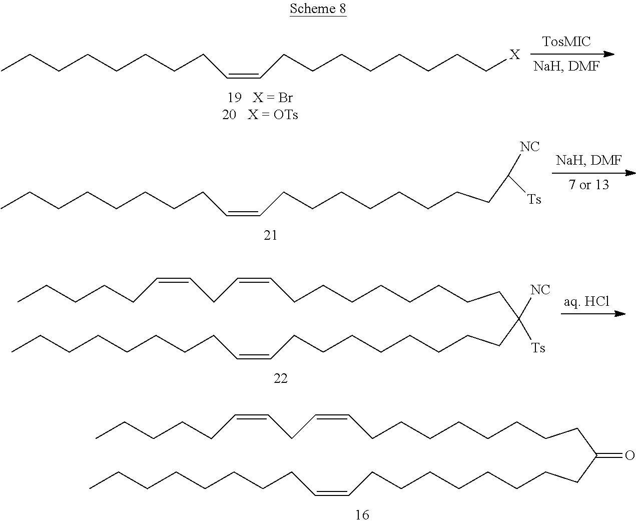

alkylated a second time with linoleyl bromide, 7, or tosylate, 13, to produce 22, which upon treatment with an aqueous solution of a strong mineral acid such as HCl, H₂SO₄, or H₃PO₄, is transformed into 16.

The mono-alkylation of TosMIC can be achieved especially advantageously in an aqueous-organic medium by the use of a base such a tetraalkylammonium hydroxide instead of the more hazardous NaH, and at a temperature between 0° C. and +100° C. For example (Scheme 9), the reaction of TosMIC with bromide 19 or tosylate 20 in a mixture of water and THF, and in the presence of tetrabutylammonium hydroxide, leads to product 21, which can then be alkylated a second time with a different alkyl halide or sulfonate, for example, linoleyl bromide, 7, or linoleyl tosylate, 13, to produce 22. The latter can then be converted into ketone 16 by the method outlined in Scheme 8.

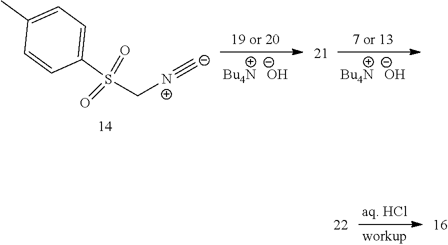

The synthesis of 17 (Scheme 10) can be achieved in a like manner starting, for example, with the mono-alkylation of TosMIC with hexadecyl bromide, 23, or tosylate 24, either in an organic solvent and in the presence of a base such as NaH, or in an aqueous-organic medium in the presence of a base such as a tetraalkylammonium hydroxide. The product of this step, 25, is alkylated a second time with linoleyl bromide, 7, or tosylate, 13, either in an organic solvent and in the presence of a base such as NaH, or in an aqueous-organic medium in the presence of a base such as a tetraalkylammonium hydroxide, to produce 26, which upon treatment with an aqueous solution of a strong mineral acid such as HCl, H₂SO₄, or H₃PO₄, is transformed into 17.

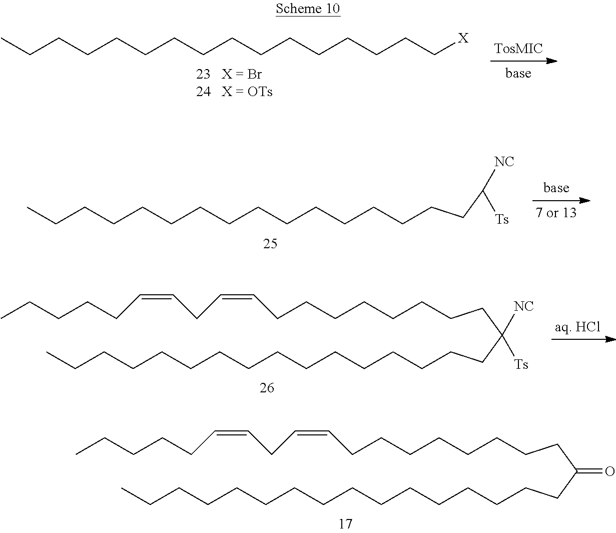

The synthesis of 18 (Scheme 11) can be achieved in a like manner starting, for example, with the mono-alkylation of TosMIC with tosylate 29 or bromide 30, either in an organic solvent and in the presence of a base such as NaH, or in an aqueous-organic medium in the presence of a base such as a tetraalkylammonium hydroxide. The product of this step, 31, is alkylated a second time with linoleyl bromide, 7, or tosylate, 13, either in an organic solvent and in the presence of a base such as NaH, or in an aqueous-organic medium in the presence of a base such as a tetraalkylammonium hydroxide, to produce 32, which upon treatment with an aqueous solution of a strong mineral acid such as HCl, H₂SO₄, or H₃PO₄, is transformed into 28. In turn, 29 and 30 can be prepared from ester 27 by reduction with, for example, lithium aluminum hydride, followed by conversion of the resulting alcohol 28 into 29 or 30 by methods that are well known to the person skilled in the art. The preparation of ester 27 and related compounds is described in a co-owned and co-pending WO 2023/215989, incorporated herein by reference.

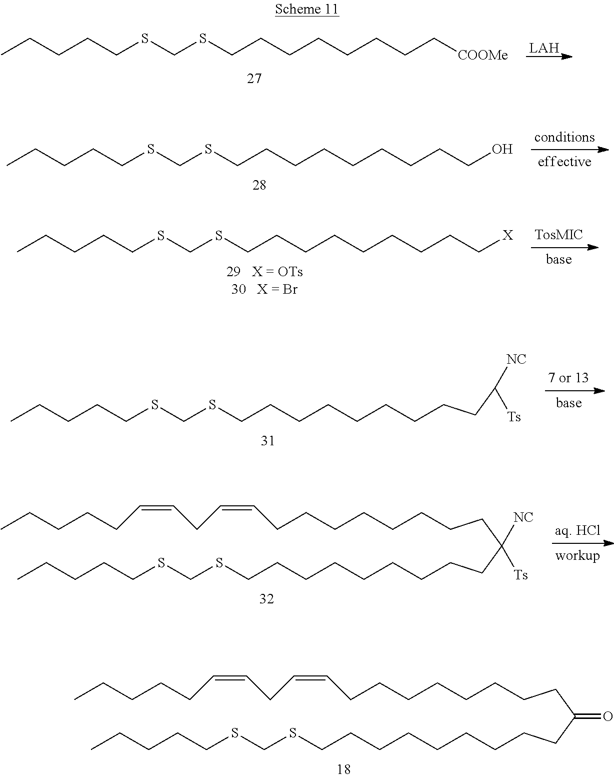

Ketones 16-18 serve as the intermediate for the synthesis of, for example, lipids 33-35 of Scheme 12:

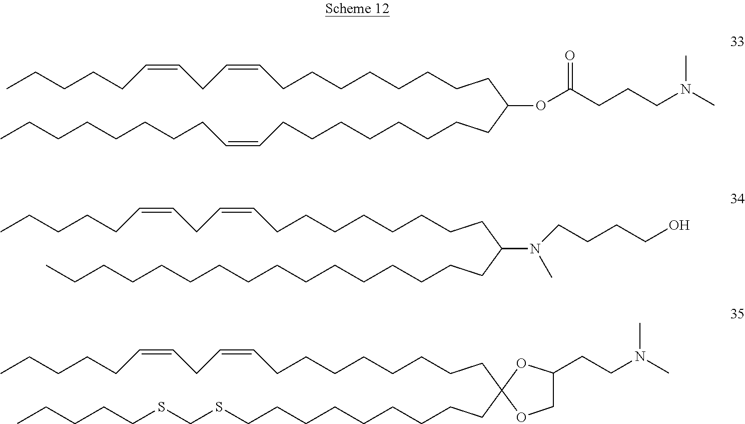

The synthesis of lipid 33 proceeds with the reduction of ketone 16 to alcohol 36, for example with a hydride reagent in an appropriate solvent. Without intending to be limiting, this step is exemplified in Scheme 13 with the conversion of 16 to 36 with NaBH4 in ethanol. Alcohol 36 is then esterified with 4-dimethylaminobutanoic acid or its hydrochloride in the presence of a condensing agent such a carbodiimide, for example, EDCI, to produce 33.

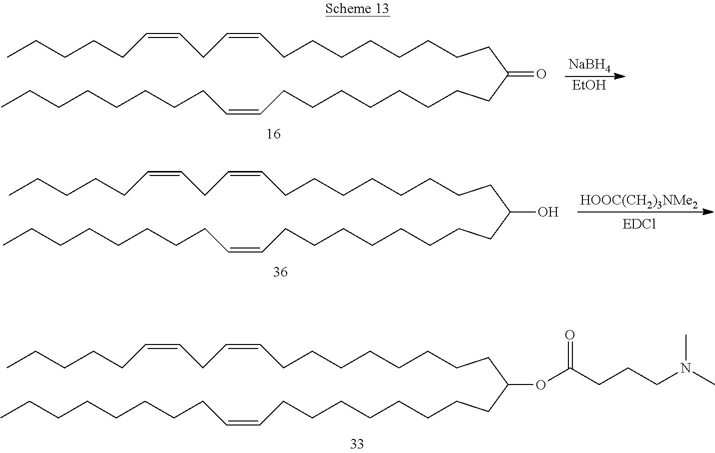

The synthesis of lipid 34 starts with the reductive amination of ketone 17 with an O-protected derivative of 4-amino-1-butanol, for example, tert-butyldiphenylsilyl ether 37, in the presence of a hydride reagent and optionally a suitable quantity of a Bronsted acid in an appropriate solvent. Without intending to be limiting, this step is exemplified in Scheme 14 with the conversion of 17 into 38 upon reaction with 37, NaBH(OAc)₃, and AcOH in dichloroethane. Secondary amine 38 is then reductively methylated, for example with aqueous formaldehyde in the presence of NaBH(OAc)₄, and product 39 is desilylated with a source of fluoride ion, for example HF-pyridine complex, to produce 34.

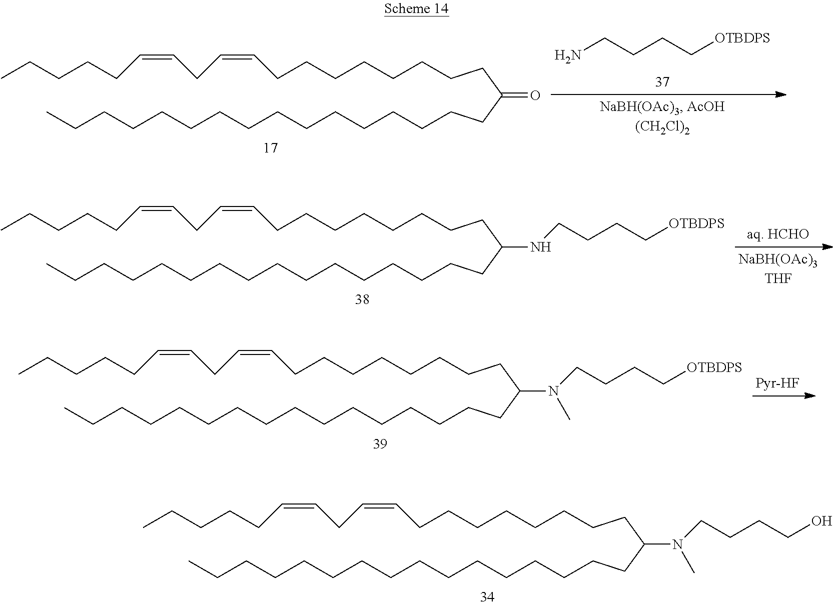

Alternatively, the conversion of 17 into lipid 34 may start with the reductive amination of the ketone with a simpler amine in the presence of a hydride reagent and optionally a suitable quantity of a Bronsted acid in an appropriate solvent. Without intending to be limiting, this step is exemplified in Scheme 15 with the conversion of 17 to 41 with methylamine and NaBH(OAc)₄ in dichloromethane. A subsequent reaction of 41 with an appropriate alcohol comprising a suitable leaving group, such as a halide or a sulfonate group, for example, 4-bromobutanol, produces lipid 36.

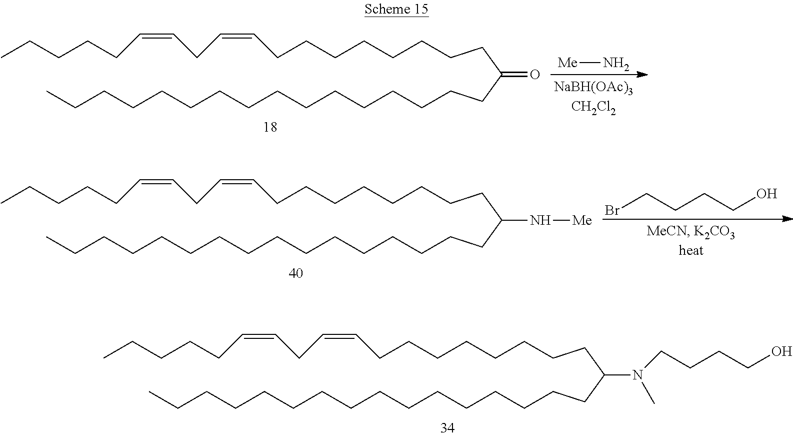

The synthesis of lipid 35 (Scheme 16) can be achieved by subjecting ketone 18 to synthetic steps analogous to those shown in Scheme 5 above for the conversion of 10 into KC2.

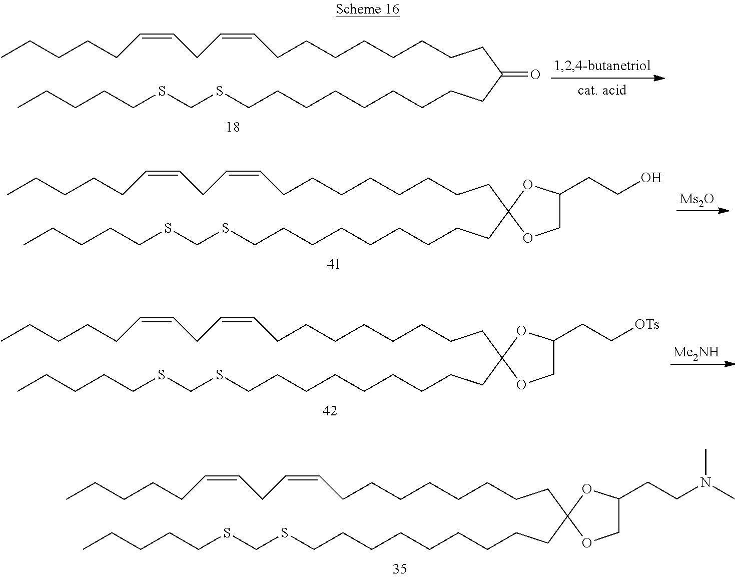 [SMILES: "Ms2O" ; "Me2NH" ; CCCCC/C=C\C/C=C\CCCCCCCCC(=O)CCCCCCCCCSCSCCCCC ; CCCCC/C=C\C/C=C\CCCCCCCCC1(CCCCCCCCCSCSCCCCC)OCC(CCO)O1 ; CCCCC/C=C\C/C=C\CCCCCCCCC1(CCCCCCCCCSCSCCCCC)OCC(CCOS(=O)(=O)c2ccc(C)cc2)O1 ; CCCCC/C=C\C/C=C\CCCCCCCCC1(CCCCCCCCCSCSCCCCC)OCC(CCN(C)C)O1]

In another embodiment, the methods of this disclosure are advantageous for the preparation of ketone building blocks for the synthesis of other types of lipids. For example, certain lipids described in co-owned and co-pending PCT/CA2023/051272 can be made from ketone 45, a synthesis of which is provided in the foregoing application. A more advantageous route to 45 involves the double alkylation of TosMIC in an aqueous-organic medium in the presence of a base such as a tetraalkylammonium hydroxide, followed by the conversion of the doubly alkylated product into a ketone by treatment with an aqueous solution of a strong mineral acid such as HCl, H₂SO₄, or H₃PO₄. Without intending to be limiting, the method is exemplified in Scheme 17 with the double alkylation of TosMIC with 6-bromo-1-hexene, 43, in the presence of tetrabutylammonium hydroxide in aqueous THF, followed by treatment of the resulting 44 with aqueous HCl.

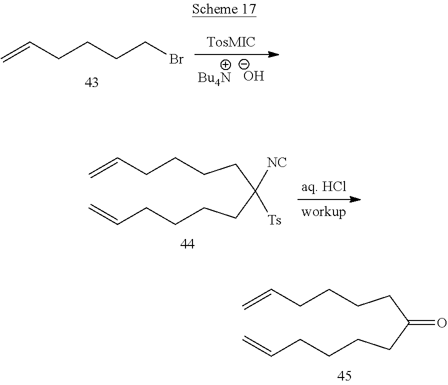

Other ketones that can be advantageously prepared by the methods of this disclosure are unsymmetrical C₁₀ to C₂₀ ketones that are building blocks for certain types of ionizable cationic lipids described in the foregoing co-owned PCT applications. One such ketone is pentadecane-7-one, 49, the synthesis of which (Scheme 18) can be achieved starting, for example, with the mono-alkylation of TosMIC with 1-bromooctane, 46, in an aqueous-organic medium in the presence of a base such as a tetraalkylammonium hydroxide; for example, in aqueous THF in the presence of tetrabutylammonium hydroxide. The product of this step, 47, is alkylated a second time with 1-bromohexane also in aqueous THF in the presence of tetrabutylammonium hydroxide, to

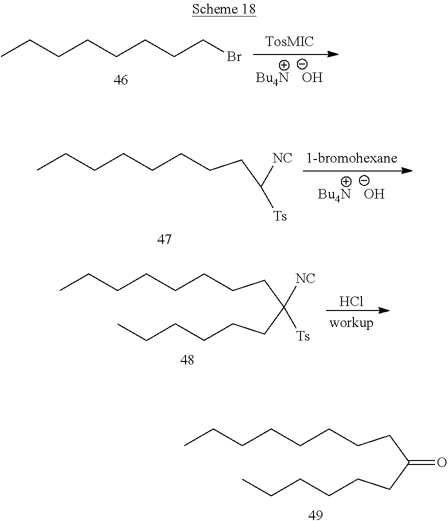

give 48, which upon treatment with an aqueous solution of a strong mineral acid such as HCl, H₂SO₄, or H₃PO₄, is transformed into 49.
The foregoing provides examples of the methods of this disclosure based on the chemistry of TosMIC. The Ogura reagent can be employed in analogous synthesis procedures, with the difference that alkylation is carried out by the use of strong bases such as NaH or KH.

Representative, but by no means limiting, examples of the use of the Ogura reagent in the preparation of symmetrical and unsymmetrical ketone building blocks for the synthesis of certain symmetrical and unsymmetrical ionizable lipids are provided below.

The dialkylation of the Ogura reagent, 50, is most advantageously carried out in the presence of a base such as potassium hydride (KH). Thus, the reaction of 50 with at least 2 molar equivalents each of an alkyl halide or sulfonate (tosylate, mesylate and the like) and KH in an appropriately inert solvent produces a dialkylated derivative. This is shown in Scheme 19 with the exemplary, but not limiting, preparation of 51 and subsequent hydrolysis thereof to symmetrical ketone 52. Those of skill in the art will recognize that the use of alternative alkyl halides or tosylates in the synthetic sequence of Scheme 19 will produce alternative symmetrical ketones. For example, the use of bromide 7 or tosylate 13 in lieu of 1-bromooctane will result in the formation of ketone 10.

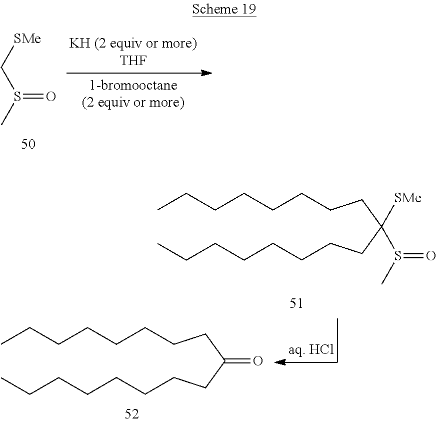

The monoalkylation of the Ogura reagent is most advantageously carried out in the presence of a base such as sodium hydride (NaH). Thus, the reaction of 50 with about 1 molar equivalent each of an alkyl halide or sulfonate (tosylate, mesylate and the like) and NaH in an appropriately inert solvent produces a monoalkyl derivative. This is shown in Scheme 20 with the exemplary, but not limiting, preparation of 53, whereby a mixture of diastereomers is obtained since the S atom of the sulfoxide group is stereogenic.

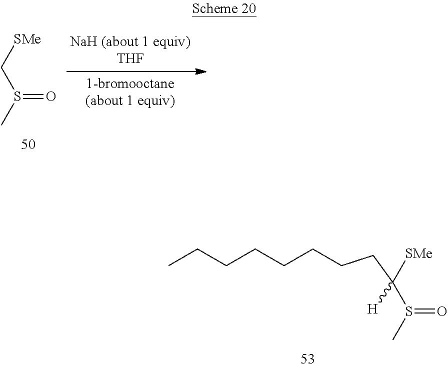

A monoalkylated derivative of the Ogura reagent such as 53, can be alkylated a second time with a different alkyl halide, leading to the formation of a dialkyl derivative. The latter can then be hydrolyzed to produce an unsymmetrical ketone. The second alkylation reaction is most advantageously carried out in the presence of a base such as potassium hydride (KH). Thus, the reaction of 53 with about 1 molar equivalent each of an alkyl halide or sulfonate (tosylate, mesylate and the like) and KH in an appropriately inert solvent produces a dialkylated derivative. This is shown in Scheme 21 with the exemplary, but not limiting, preparation of 54 and subsequent hydrolysis thereof to unsymmetrical ketone 55. Those of ordinary skill in the art will recognize that the use of alternative alkyl halides or tosylates in the synthetic sequences of Schemes 20 and 21 will produce alternative unsymmetrical ketones.

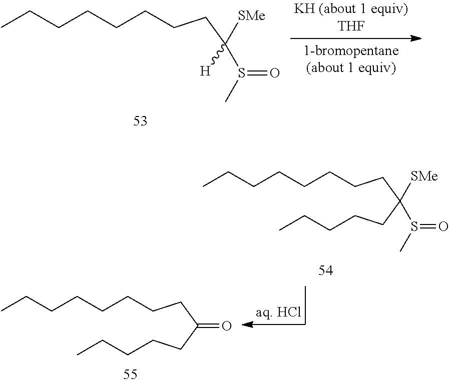 [SMILES: CCCCCCCCC(SC)S(C)=O ; CCCCCCCCC(CCCCC)(SC)S(C)=O ; CCCCCCCCC(=O)CCCCC]

The ionizable lipid produced by the method of the disclosure may be formulated in a variety of delivery vehicles known to those of ordinary skill in the art. An example of a delivery vehicle is a lipid nanoparticle, which includes liposomes, lipoplexes, polymer nanoparticles comprising lipids, polymer-based nanoparticles, emulsions, and micelles.

In one embodiment, the ionizable lipids are formulated in a delivery vehicle by mixing them with additional lipids, including helper lipids, such as vesicle forming lipids and optionally an aggregation inhibiting lipid, such as a hydrophilic polymer-lipid conjugate (e.g., PEG-lipid).

As set forth previously, a helper lipid includes a sterol, a diacylglycerol, a ceramide or derivatives thereof.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol, and the like.

Examples of diacylglycerols include dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof. These lipids may be synthesized or obtained from natural sources, such as from egg.

A suitable ceramide derivative is egg sphingomyelin.

Delivery vehicles incorporating the ionizable lipids can be prepared using a wide variety of well described formulation methodologies known to those of skill in the art, including but not limited to extrusion, ethanol injection and in-line mixing. Such methods are described in Maclachlan, I. and P. Cullis, “Diffusible-PEG-lipid Stabilized Plasmid Lipid Particles”, Adv. Genet., 2005. 53PA: 157-188; Jeffs, L. B., et al., “A Scalable, Extrusion-free Method for Efficient Liposomal Encapsulation of Plasmid DNA”, Pharm Res, 2005. 22(3): 362-72; and Leung, A. K., et al., “Lipid Nanoparticles Containing siRNA Synthesized by Microfluidic Mixing Exhibit an Electron-Dense Nanostructured Core”, The Journal of Physical Chemistry. C, Nanomaterials and Interfaces, 2012, 116(34): 18440-18450, each of which is incorporated herein by reference in its entirety.

The delivery vehicle can also be a nanoparticle that is a lipoplex that comprises a lipid core stabilized by a surfactant. Vesicle-forming lipids may be utilized as stabilizers. The lipid nanoparticle in another embodiment is a polymer-lipid hybrid system that comprises a polymer nanoparticle core surrounded by stabilizing lipid.

Nanoparticles comprising the ionizable lipid may alternatively be prepared from polymers without lipids. Such nanoparticles may comprise a concentrated core of a therapeutic agent that is surrounded by a polymeric shell or may have a solid or a liquid dispersed throughout a polymer matrix.

The following examples are given for the purpose of illustration only and not by way of limitation on the scope of the invention.

### EXAMPLES

### Organic Synthesis of Lipids 1 (KC2) and 2 (MC3)

Unless otherwise specified, all reagents and solvents were commercial products and were used without further purification, except THF (freshly distilled from Na/benzophenone under Ar), CH₂Cl₂ (freshly distilled from CaH₂ under Ar). “Dry methanol” was freshly distilled from magnesium turnings. All reactions were performed under an argon atmosphere. Reaction mixture from aqueous workups were dried by passing over a plug of anhydrous Na₂SO₄ held in a filter tube and concentrated under reduced pressure on a rotary evaporator. Thin-layer chromatography was performed on silica gel plates coated with silica gel (Merck™ 60 F254 plates) and column chromatography was performed on 230-400 mesh silica gel. Visualization of the developed chromatogram was performed by staining with I₂ or potassium permanganate solution. ¹H and ¹³C nuclear magnetic resonance (NMR) spectra were recorded at room temperature in CDCl₃ solutions. ¹H NMR spectra were referenced to residual CHCl₃ (7.26 ppm) and ¹³C NMR spectra were referenced to the central line of the CDCl₃ triplet (77.00 ppm). Chemical shifts are reported in parts per million (ppm) on the δ scale. Multiplicities are reported as “s” (singlet), “d” (doublet), “t” (triplet), “q” (quartet), “m” (multiplet), and further qualified as “app” (apparent) and “br” (broad). Low- and high-resolution mass spectra (m/z) were obtained in the electrospray (ESI) and field desorption/field ionization (FD/FI) mode.

### Example 1: Production of Symmetrical Ionizable Lipids and Symmetrical Ketone Building Blocks for Ionizable Lipids by Dialkylation of TosMIC

(a) Representative procedure for the dialkylation of TosMIC in the presence of NaH: 1-(((6Z,9Z,28Z,31Z)-19-isocyanoheptatriaconta-6,9,28,31-tetraen-19-yl)sulfonyl)-4-methylbenzene (15). A solution of TosMIC (5.57 g, 28.53 mmol, 1.2 equiv) in extremely dry N,N-dimethylformamide (DMF, 10 mL) was added over 30 min

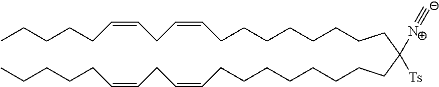 [SMILES: [C-]#[N+]C(CCCCCCCC/C=C\C/C=C\CCCCC)(CCCCCCCC/C=C\C/C=C\CCCCC)S(=O)(=O)c1ccc(C)cc1]

(syring pump) to a cold (0° C.), vigorously stirred solution of linoleyl tosylate (13, 20 g, 47.55 mmol, 2 equiv.) in extremely dry DMF (15 mL) containing suspended sodium hydride (60% wt. in oil, 2.38 g, 59.44 mmol, 2.5 equiv), under nitrogen. The resulting mixture was allowed to warm up to room temperature, stirred for 2 hr, diluted with hexanes (50 mL), and carefully poured into vigorously stirred, ice-cold, aqucous saturated ammonium chloride solution. The organic layer was separated and the aqueous layer was extracted with n-hexane (2×50 mL). The combined extracts were washed with brine, dried (Na₂SO₄), filtered, and concentrated in vacuo. The residue was eluted through a plug of basic alumina with 1:9 ethyl acetate-n-hexane to afford 15 (16.46 g, 23.78 mmol, ˜100%) as a colorless oil, which was used for the next step without further purification. ¹H NMR (400 MHZ, CDCl₃): δ7.86 (d, J=8.3 Hz, 2H), 7.41 (d, J=8.1 Hz, 2H), 5.45-5.19 (m, 8H), 2.77 (t, J=6.3 Hz, 4H), 2.48 (s, 3H), 2.11-1.82 (m, 12H), 1.63-1.21 (m, 36H), 0.89 (t, J=6.8 Hz, 6H). ¹³C NMR (100 MHZ, CDCl₃) δ163.6, 146.2, 131.1, 130.2, 130.0, 129.8, 128.0, 127.9, 81.9, 33.1, 31.5, 29.6, 29.4, 29.4, 29.3, 29.2, 27.2, 25.6, 23.6, 22.6, 21.8, 14.1. The following compound was prepared by the same method:
1-((7-isocyanotrideca-1,12-dien-7-yl)sulfonyl)-4-methylbenzene (44). From TosMIC

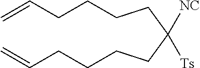

and 6-bromo-1-hexene. ¹H NMR (400 MHZ, CDCl₃): δ7.86 (d, J=8.3 Hz, 2H), 7.41 (d, J=8.1 Hz, 2H), 5.73 (m, 2H), 4.92 (m, 4H), 2.47 (s, 3H), 2.12-1.22 (m, 16H).

(b) Representative procedure for the dialkylation of TosMIC in the presence of Bu₄NOH: 1-((7-isocyanotrideca-1,12-dien-7-yl)sulfonyl)-4-methylbenzene (44). To a solution of tert-butyl ammonium hydroxide 40% wt. in water (15 g, 23 mmol, 3 equiv.) and 6-bromo-1-hexene (3 g, 18.4 mmol, 2 equiv.) in THF (5 mL), a solution of TosMIC (1.8 g, 18.4 mmol, 1 equiv) in THF (3 mL) was added dropwise using a syringe pump over 30 min. The mixture and stirred for 8 hr, then it was cooled down to 0° C. and neutralized to pH 7 with 6N aqueous HCl. The neutral mixture was extracted with diethyl ether (3×15 mL). The combined extracts were washed with brine, dried (Na₂SO₄) and concentrated in vacuo. The crude residue (3.3 g, ˜quant.) was used directly in the next step. NMR data are as provided in part (a). The following compound was prepared by a similar method: 1-(((6Z,9Z,28Z,31Z)-19-isocyanoheptatriaconta-6,9,28,31-tetraen-19-yl)sulfonyl)-4-methylbenzene (15). From TosMIC and linoleyl tosylate, 13, except that the reaction was carried out at 50° C. instead of room temperature. NMR data are as provided in part (a).

(c) Representative procedure for the hydrolysis of a dialkylated

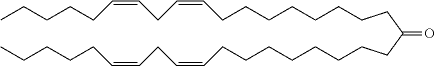 [SMILES: CCCCC/C=C\C/C=C\CCCCCCCCC(=O)CCCCCCCC/C=C\C/C=C\CCCCC]

TosMIC derivative to a ketone: (6Z,9Z,28Z,31Z)-heptatriaconta-6,9,28,31-tetraen-19-one (10). Concentrated aqucous HCl solution (12M, 3.6 mL, 43.4 mmol, 6 equiv.) was added dropwise to a cold (0° C.) solution of 1-(((6Z,9Z,28Z,31Z)-19-isocyanoheptatriaconta-6,9,28,31-tetraen-19-yl)sulfonyl)-4-methylbenzene (15, 5 g, 7.23 mmol, 1.0 equiv.) in ether (10 mL) and the mixture was stirred for 15 min at the same temperature. The mixture was then diluted with ether (30 mL) and slowly poured into a vigorously stirred, ice-cold, saturated aqueous NaHCO₃ solution. The organic layer was separated, and the aqueous layer was further extracted with ether (2×20 mL). The combined extracts were washed with brine, dried (Na₂SO₄), filtered, and concentrated in vacuo to afford 10 (3.81 g, 7.23 mmol, ˜100%) as a colorless oil, which was used for the next step without further purification. ¹H NMR (400 MHZ, CDCl₃): δ5.32 (m, 8H), 2.74 (t, 4H), 2.35 (t, 4H), 2.02 (m, 8H), 1.55-1.20 (m, 36H), 0.87 (t, 6H). ¹³C NMR (100 MHZ, CDCl₃): δ210.9, 130.0, 129.8, 128.0, 127.8, 42.6, 31.4, 29.7, 29.5, 29.24, 29.22, 29.1, 29.0, 27.0 (2 overlapping peaks), 25.5, 23.7, 22.5, 14.0. LRMS m/z 527 [M+H]⁺, 549 [M+Na]⁺. The following compound was prepared by the same method: trideca-1,12-dien-7-one (45). From 44. ¹H NMR (300 MHZ, CDCl₃): δ5.73 (m, 2H), 4.92

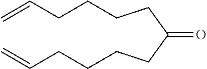

(m, 4H), 2.35 (t, 4H, J=7.3 Hz), 2.01-1.28 (12H), ¹³C NMR (75 Mhz, CDCl₃): δ210.6, 138.3, 114.5, 42.5, 33.5, 28.4, 23.2.

(d) Representative procedure for the in situ hydrolysis of dialkylated TosMIC derivatives: trideca-1,12-dien-7-one (45). A solution of TosMIC (3.6 g, 18.4

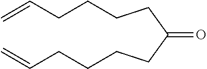

mmol, 1 equiv) in THF (5 mL) was added dropwise over 30 min (syringe pump) to a vigorously stirred solution of tetrabutylammonium hydroxide (40% wt. in water, 30 g, 46 mmol, 3 equiv.) and 6-bromo-1-hexene (6 g, 36.8 mmol, 2 equiv.) in THF (10 mL), at room temperature and under nitrogen. The mixture was stirred for 8 h, then it was cooled down to 0° C. Aqueous concentrated HCl (12M, 9.2 mL, 110.4 mmol, 6 equiv.) was carefully added dropwise. The mixture was stirred for 15 min at the same temperature, then it was diluted with ether (30 mL) and carefully poured into vigorously stirred, ice-cold, saturated aqueous NaHCO₃ solution. The organic layer was separated, and the aqueous layer was further extracted with ether (2×30 mL). The combined extracts were washed with brine (sat. solution), dried (Na₂SO₄), filtered, and concentrated in vacuo to afford crude 47 (3.58 g, 18.4 mmol, ˜100%) as a colorless oil and used for the next step without further purification. NMR data are as provided in part (c). The following compound was prepared by the same method:

(6Z,9Z,28Z,31Z)-heptatriaconta-6,9,28,31-tetraen-19-one (10). From TosMIC and linoleyl tosylate, 13. NMR data are as provided in Part (c).

(e) 2-(2,2-di((9Z,12Z)-Octadeca-9,12-dien-1-yl)-1,3-dioxolan-4-yl)ethan-1-ol (11). A mixture of (6Z,9Z,28Z,31Z)-heptatriaconta-6,9,28,31-tetraen-19-one

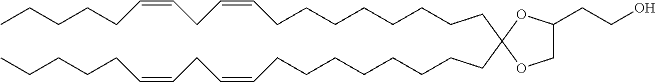 [SMILES: CCCCC/C=C\C/C=C\CCCCCCCCC1(CCCCCCCC/C=C\C/C=C\CCCCC)OCC(CCO)O1]

(10, 527 mg, 1.0 mmol), 1,2,4-butanetriol (technical grade, ca. 90%, 236 mg, 2 mmol) and pyridinium p-toluenesulfonate (50 mg, 0.2 mmol) in 50 mL of toluene was refluxed under nitrogen overnight with continuous removal of water (Dean-Stark trap). Upon completion of the reaction, the mixture was cooled to room temperature, washed with water (2×30 mL), dried by passing over a plug of anhydrous Na₂SO₄ and evaporated. The yellowish oily residue (0.6 g) was purified by silica gel (230-400 mesh, 50 g) column chromatography, with dichloromethane as eluent, to afford 0.87-0.93 mmol (87-93%) of pure ketal. ¹H NMR (300 MHz, CDCl₃) δ5.36 (m, 8H), 4.24 (m, 1H), 4.09 (m, 1H), 3.81 (m, 2H), 3.53 (t, 1H), 2.78 (t, 4H), 2.21 (t, 1H [OH]), 2.05 (m, 8H), 1.82 (m, 2H), 1.65-1.53 (m, 4H), 1.42-1.23 (m, 36H), 0.89 (t, 6H). ¹³C NMR (75 MHz, CDCl₃; some signals are doubled) 8 130.2, 130.1, 128.0, 127.9, 112.6, 75.5, 69.9, 60.9, 37.8, 37.3, 35.4, 31.5, 29.9, 29.7, 29.5, 29.34, 29.27, 27.22, 27.19, 25.6, 24.0, 23.8, 22.6, 14.1. LRMS: m/z 615 [M+H]⁺, 637 [M+Na]⁺.

(f) 2-(2,2-di((9Z,12Z)-Octadeca-9,12-dien-1-yl)-1,3-dioxolan-4-yl)ethyl methane-sulfonate (12). Neat methanesulfonic anhydride (290 mg, 1.6 mmol) was added

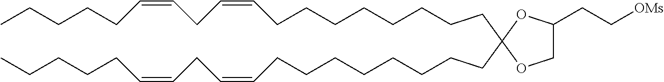 [SMILES: CCCCC/C=C\C/C=C\CCCCCCCCC1(CCCCCCCC/C=C\C/C=C\CCCCC)OCC(CCOS(C)(=O)=O)O1]

to a solution of 2-(2,2-di((9Z,12Z)-octadeca-9,12-dien-1-yl)-1,3-dioxolan-4-yl)ethan-1-ol (0.8 mmol) and dry triethylamine (242 mg, 330 uL, 2.4 mmol) in 5 mL of dry CH₂Cl₂. The resulting mixture was stirred at room temperature overnight. The mixture was diluted with 25 mL of CH₂Cl₂. the organic phase was washed with water (2×30 mL), passed over a plug of anhydrous Na₂SO₄, and evaporated to afford 510 mg of mesylate as yellowish oil. The crude mesylate was used in the following step without further purification. ¹H NMR (300 MHz, CDCl₃) δ5.34 (m, 8H), 4.35 (m, 2H), 4.18 (m, 1H), 4.08 (m, 1H), 3.52 (t, 1H), 3.01 (s, 3H), 2.76 (t, 4H), 2.01 (m, 10H), 1.58-1.20 (m, 40H), 0.88 (t, 6H). ¹³C NMR (75 MHz, CDCl₃, some signals are doubled) δ130.1, 130.0, 127.9, 127.8, 112.6, 72.2, 69.5, 67.1, 37.6, 33.3, 31.5, 31.4, 29.83, 29.81, 29.6, 29.5, 29.3, 29.21, 29.19, 27.2, 27.1, 25.6, 24.0, 23.7, 22.5, 14.0. LRMS: m/z 665 [M+H]⁺, 687 [M+Na]⁺.

(g) 2-(2,2-di((9Z,12Z)-Octadeca-9,12-dien-1-yl)-1,3-dioxolan-4-yl)-N,N-dimethyl-ethan-1-amine (1, KC2). The above crude mesylate (500 mg) was added to 20

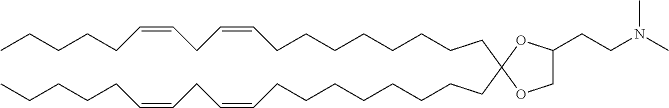 [SMILES: CCCCC/C=C\C/C=C\CCCCCCCCC1(CCCCCCCC/C=C\C/C=C\CCCCC)OCC(CCN(C)C)O1]

mL of a commercial 2.0 M solution of dimethylamine in THF. The resulting mixture was stirred at room temperature for 6 days, whereupon no more mesylate was apparent by TLC and/or ¹H NMR. Evaporation of the volatiles returned an oily residue that was purified by column chromatography on silica gel (230-400 mesh, 50 g) with 0-5% methanol gradient in dichloromethane as eluent, resulting in recovery of 350-400 mg of pure product. ¹H NMR (300 MHz, CDCl₃) δ5.36 (m, 8), 4.07 (m, 2H), 3.49 (t, 1H), 2.78 (t, 4H), 2.46-2.24 (m, 2H), 2.23 (s, 6H), 2.06 (m, 8H), 1.89-1.59 (m, 2H), 1.58 (m, 4H), 1.42-1.20 (m, 36H), 0.90 (br t, 6H). ¹³C NMR (75 MHz, CDCl₃; some signals are doubled) δ130.1, 127.9, 112.1, 74.7, 69.9, 56.3, 45.4, 37.8, 37.5, 31.8, 31.5, 29.9, 29.7, 29.6, 29.5, 29.3, 27.2, 25.6, 24.0, 23.7, 22.6, 14.1. LRMS: m/z 642 [M+H]⁺.

(h) (6Z,9Z,28Z,31Z)-Heptatriaconta-6,9,28,31-tetraen-19-ol (9). Solid NaBH4 (2 mmol) was added portionwise to a cold (0° C.) stirred solution of

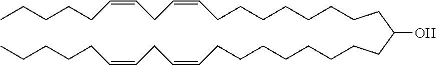 [SMILES: CCCCC/C=C\C/C=C\CCCCCCCCC(O)CCCCCCCC/C=C\C/C=C\CCCCC]

(6Z,9Z,28Z,31Z)-heptatriaconta-6,9,28,31-tetraen-19-one (10, 2 mmol) in 95% ethanol (10 mL). After stirring at 0° C. for 1 h, the reaction was checked for completion, either by TCL (5% ether in hexanes) or, more reliably, by adding 3-4 drops of the reaction mixture to saturated aqueous NH₄Cl solution (0.5 mL), extracting with hexanes, evaporating the combined extracts to dryness, and checking the residue by ¹H NMR. Either method indicated that the reaction was complete. The reaction was quenched by careful addition of aqueous saturated NH₄Cl solution (caution should be taken due to H₂ evolution and foaming) and concentrated on the rotary evaporator to remove the ethanol. The aqueous residue was extracted with hexanes (3×10 mL). The combined extracts were passed through a plug of anhydrous Na₂SO₄ and concentrated to afford crude alcohol, which was purified by silica gel column chromatography with 5→10% v/v ethyl acetate in hexanes (91% yield). ¹H NMR (300 MHz, CDCl₃) δ5.34 (m, 8H), 3.57 (m, 1H), 2.77 (t, J=6.2 Hz, 4H), 2.05 (q, J=6.7 Hz, 8H), 1.56-1.15 (m, 40H), 0.99 (t, J=6.2 Hz, 6H). ¹³C NMR (75 MHz, CDCl₃) δ130.1, 130.1, 127.9, 127.93, 127.9, 71.90, 37.5, 31.5, 29.7, 29.64, 29.6, 29.5, 29.32, 29.3, 27.2, 27.17, 25.64, 25.6, 22.5, 14.0. LRMS: m/z 529 [M+H]+, 551 [M+Na]⁺.

(6Z,9Z,28Z,31Z)-heptatriaconta-6,9,28,31-tetraen-19-yl 4-(dimethylamino)-butanoate (2, MC3). A solution of (6Z,9Z,28Z,31Z)-heptatriaconta-6,9,28,31-tetraen-19-ol (9, 1 mmol, 1.0 equiv), 4-dimethylaminobutyric acid

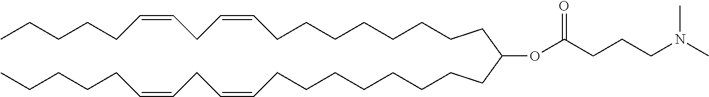 [SMILES: CCCCC/C=C\C/C=C\CCCCCCCCC(CCCCCCCC/C=C\C/C=C\CCCCC)OC(=O)CCCN(C)C]

hydrochloride (1.2 mmol, 1.2 equiv), diisopropylethylamine (1.5 mmol, 1.5 equiv), and DMAP (0.1 mmol, 0.1 equiv) in dry CH₂Cl₂ (3 mL) was stirred at room temperature for 5 minutes prior to the addition of EDCI (1.5 mmol, 1.5 equiv). The mixture was stirred overnight at room temperature, under argon, whereupon TLC (5% MeOH in CH₂Cl₂) and ¹H NMR indicated that the reaction had completed. The solution was diluted with more CH₂Cl₂ (10 mL) and sequentially washed with aqueous saturated NaHCO₃ (5 mL) and water (10 mL). The organic phase was passed over a plug of anhydrous Na₂SO₄ and concentrated in vacuo. The residue of crude product was purified by flash column chromatography with 3% v/v MeOH in CH₂Cl₂, containing 0.1% NEt₃ (78% yield). ¹H NMR (300 MHz, CDCl₃) δ5.33 (m, 8H), 4.85 (m, 1H), 2.75 (t, 4H), 2.40 (m, 2H), 2.34 (t, 2H), 2.20 (s, 6H), 2.02 (m, 8H), 1.77 (m, 2H), 1.50 (m, 6H), 1.39-1.17 (m, 34H), 0.87 (t, 6H). ¹³C NMR (75 MHz, CDCl_{3;} some signals are doubled) δ173.3, 130.1, 130.0, 127.9, 127.8, 74.1, 59.0, 45.4, 34.1, 32.4, 31.5, 29.7, 29.6, 29.5, 29.4, 29.33, 29.30, 29.2, 27.15, 27.14, 25.3, 23.1, 14.8. LRMS: m/z 642 [M+H]⁺.

### Example 2: Production of Unsymmetrical Ionizable Lipids and Unsymmetrical Ketone Building Blocks for Ionizable Lipids by Stepwise Alkylation of TosMIC

Representative procedure for the monoalkylation of TosMIC in the presence of NaH: (Z)-1-((1-isocyanononadec-10-en-1-yl)sulfonyl)-4-methylbenzene (21). A solution

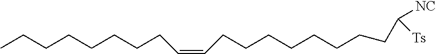

of TosMIC (200 mg, 1.0 mmol, 1 eq.) in dry DMF (1.0 mL) was added dropwise to a cold (0° C.), well-stirred suspension of NaH (60% dispersion in mineral oil, 45 mg, 1.1 mmol, 1.1 eq.) in dry DMF (0.22 mL) maintained under nitrogen atmosphere. After stirring for 10 minutes, a solution of oleyl tosylate (423 mg, 1 mmol) in dry DMF (1 mL) was added dropwise to the mixture via syringe. The solution was allowed to warm to room temperature and stirred for 5 hours, then it was cooled back to 0° C., cautiously quenched with water (3 mL), and diluted with ethyl acetate (5 mL). The layers were separated, the organic phase was collected, and the aqueous layer was extracted with ethyl acetate (2×5 mL). The combined organic phases were washed with water (1×10 mL), dried (Na₂SO₄), filtered and evaporated. The residue was purified by silica gel chromatography (0 to 20% EtOAc in hexanes) to yield 320 mg (72%) of pure 21. Some dialkylated product was also isolated during the chromatography. ¹H NMR (400 MHZ, CDCl₃) δ7.85 (d, J=8.4 Hz, 2H), 7.42 (d, J=8.0 Hz, 2H), 5.38 (m, 2H), 4.46 (dd, J=11.0, 3.4 Hz, 1H), 2.48 (s, 3H), 2.16-1.82 (m, 6H), 1.63-1.21 (m, 24H), 0.89 (t, J=6.8 Hz, 3H). The following compounds were prepared by the same procedure:

1-((1-isocyano-heptadecyl)sulfonyl)-4-methylbenzene (25). Obtained in

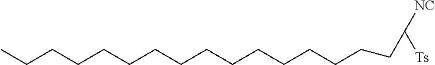

60% yield from TosMIC and palmityl tosylate.¹H NMR (400 MHZ, CDCl₃) δ7.85 (d, J=8.4 Hz, 2H), 7.42 (d, J=8.0 Hz, 2H), 4.46 (dd, J=11.0, 3.4 Hz, 1H), 2.47 (s, 3H), 2.16 (m, 1H), 1.82 (m, 1H), 1.69-1.38 (m, 2H), 1.36-1.17 (m, 26H), 0.87 (t, J=6.8 Hz, 3H).

10-isocyano-10-tosyldecyl)((pentylthio)methyl)sulfane (31). Obtained in

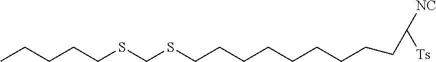

64% yield from TosMIC and tosylate 29. ¹H NMR (400 MHZ, CDCl₃) δ7.85 (d, J=8.4 Hz, 2H), 7.42 (d, J=8.0 Hz, 2H), 4.46 (dd, J=11.0, 3.4 Hz, 1H), 3.67 (s, 2H), 2.63 (br t, 4H), 2.47 (s, 3H), 2.15-1.83 (m, 2H), 1.60-1.21 (m, 20H), 0.92 (t, 3H).

(b) Representative procedure for the alkylation of a monoalkyl derivative of TosMIC in the presence of NaH: 1-(((6Z,9Z,28Z)-19-isocyanoheptatriaconta-6,9,28-trien-19-yl)sulfonyl)-4-methylbenzene (22). A solution of 21 (116 mg, 0.26 mmol, 1 equiv) in

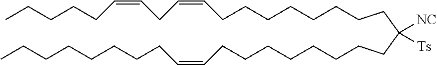

dry DMF (0.1 mL) was added dropwise to a cold (0° C.), well stirred suspension of NaH (60% dispersion in mineral oil, 25 mg, 0.62 mmol, 2 equiv) in dry DMF (0.2 mL) maintained under nitrogen atmosphere. After stirring for 10 minutes, a solution of linoleyl tosylate (118 mg, 0.28 mmol, 1.1 eq.) in dry DMF (0.3 mL) was added dropwise to the mixture via syringe. The solution was allowed to warm to room temperature and stirred for 1 hour, then it was cooled back to 0° C., cautiously quenched with water (3 mL), and diluted with hexanes (5 mL). The organic layer was collected and the aqueous phase was extracted with hexanes (2×5 mL). The combined extracts were dried (Na₂SO₄), filtered, and evaporated. The residue was purified by automated flash chromatography (0-7% EtOAc in hexanes) to yield 22 (97 mg, 0.14 mmol, 53%). ¹H NMR (400 MHZ, CDCl₃): δ7.86 (d, J=8.3 Hz, 2H), 7.41 (d, J=8.1 Hz, 2H), 5.45-5.19 (m, 6H), 2.77 (t, J=6.3 Hz, 2H), 2.48 (s, 3H), 2.11-1.82 (m, 12H), 1.63-1.21 (m, 42H), 0.89 (br t, J=6.8 Hz, 6H). The same procedure was used to prepare the following compounds:

1-(((26Z,29Z)-17-isocyanopentatriaconta-26,29-dien-17-yl)sulfonyl)-4-methylbenzene (26). Obtained in 43% yield from 25 and linoleyl tosylate. ¹H NMR (400

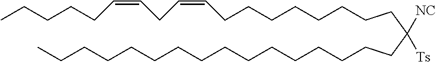

MHz, CDCl₃) δ7.86 (d, J=8.3 Hz, 2H), 7.41 (d, J=8.1 Hz, 2H), 5.43-5.28 (m, 4H), 2.77 (t, J=6.5 Hz, 2H), 2.48 (s, 3H), 2.16-1.80 (m, 6H), 2.05 (q, J=6.8 Hz, 4H), 1.59-1.16 (m, 44H), 0.90 (br t, 6H).

((19Z,22Z)-10-isocyano-10-tosyloctacosa-19,22-dien-1-yl)((pentylthio)methyl)sulfane 32). Obtained in 50% yield from 31 and linoleyl tosylate.

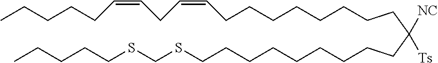

¹H NMR (400 MHZ, CDCl₃) δ7.85 (d, J=8.4 Hz, 2H), 7.42 (d, J=8.0 Hz, 2H), 5.43-5.28 (m, 4H), 3.67 (s, 2H), 2.77 (t, J=6.5 Hz, 2H), 2.63 (br t, 4H), 2.48 (s, 3H), 2.17-1.80 (m, 8H), 1.60-1.21 (m, 38H), 0.91 (br t, 6H).

(c) Representative procedure for the monoalkylation of TosMIC in the presence of Bu4NOH: 1-((1-isocyanononyl)sulfonyl)-4-methylbenzene (47). A solution of

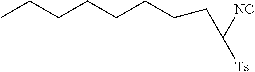

Bu₄NOH (40% wt. in water, 1.66 g, 2.56 mmol, 1 equiv.) was added in one portion to a vigorously stirred solution of n-bromooctane (494 mg, 2.56 mmol, 1 equiv) and TosMIC (500 mg, 2.56 mmol, 1 equiv) in THF at room temperature and under nitrogen. The mixture was stirred for 1 h, then it was diluted with ether (30 mL). The organic layer was separated and the aqueous phase was further extracted with ether (2×30 mL). The combined extracts were washed with brine, dried (Na₂SO₄), filtered, and concentrated in vacuo. The residue was purified by flash column chromatography (1:9 ethyl acetate:hexanes) to afford the title compound (598 mg, 1.94 mmol, 76%) as a yellow solid, m.p 45.2-46.5° C. ¹H NMR (300 MHz, CDCl₃): δ7.85 (d, 2H, J 8.4 Hz), 7.41 (d, 2H, J=8.1 Hz), 4.42 (dd, 1H, J=10.9, 3.4 Hz), 2.47 (s, 3H), 2.16 (m. 1H), 1.81 (m, 1H), 1.60-1.26 (m, 12H), 0.87 (t, 3H, J=7.3 Hz). ¹³C NMR (75 MHz, CDCl₃): δ164.8, 146.6, 131.2, 130.1, 130.1, 73.0, 31.7, 29.1, 29.1, 28.6, 28.4, 25.4, 22.6, 21.8, 14.1.

(d) Representative procedure for the alkylation of a monoalkyl derivative of TosMIC in the presence of BuNOH and hydrolysis of the resulting product: 7-pentadecanone (49). A solution of 1-((1-isocyanononyl)sulfonyl)-4-methylbenzene (598 mg, 1.94 mmol, 1 equiv) in THF (2 mL) was added to a solution of 1-bromohexane (320 mg, 1.94 mmol, 1 equiv) and Bu4NOH 40% wt. in water (2.52 g, 3.88 mmol, 2 equiv.) in THF (5 mL) at room temperature and under nitrogen. The mixture was stirred for 8 hr, then it was cooled to 0° C. Aqueous concentrated HCl (12M, 1 mL, 11.64 mmol, 6 equiv.) was added dropwise, and the solution was allowed to warm to room temperature and stirred for 15 min. The mixture was carefully transferred into a cold (0° C.), vigorously stirred, aqueous saturated NaHCO₃ solution and the aqueous layer was extracted with ether (3×25 mL). The combined extracts were washed with brine, dried (Na₂SO₄), filtered, and concentrated in vacuo to afford 49 (439 mg, 1.94 mmol, ˜quant) as a colorless oil that required no further purification. ¹H NMR (300 MHz, CDCl₃): δ2.40 (t, 4H, t, J=7.4 Hz), 1.58-1.28 (m, 20H), 0.89 (br t, 6H). ¹³C NMR (75 MHZ, CDCl₃): 8 211.8, 42.8, 31.8, 31.6, 29.4, 29.3, 29.1, 29.0, 23.9, 23.8, 22.6, 22.5, 14.1, 14.0.

(c) Representative procedure for the conversion of a doubly alkylated TosMIC into a ketone: (6Z,9Z,28Z)-heptatriaconta-6,9,28-trien-19-one (16). Compound

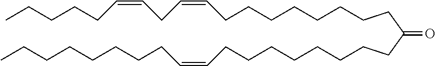 [SMILES: CCCCC/C=C\C/C=C\CCCCCCCCC(=O)CCCCCCCC/C=C\CCCCCCCC]

22 was converted into ketone 16 in 73% yield by the procedure described above for the synthesis of ketone 10. ¹H NMR (400 MHZ, CDCl₃) δ5.32 (m, 6H), 2.74 (t, 2H), 2.35 (t, 4H), 2.02 (m, 8H), 1.56-1.19 (m, 42H), 0.87 (br t, 6H).

(26Z,29Z)-pentatriaconta-26,29-dien-17-one (17). Compound 26 was

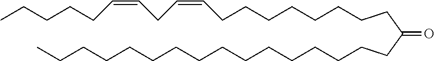 [SMILES: CCCCC/C=C\C/C=C\CCCCCCCCC(=O)CCCCCCCCCCCCCCCC]

converted into ketone 17 in 73% yield by the procedure described above for the synthesis of ketone 10. ¹H NMR (400 MHz, CDCl₃) δ5.43-5.28 (m, 4H), 2.74 (t, 2H), 2.36 (br t, 4H), 2.02 (m, 4H), 1.58-1.20 (m, 46H), 0.87 (br t, 6H).

(19Z,22Z)-1-(((pentylthio)methyl)thio)octacosa-19,22-dien-10-one (18).

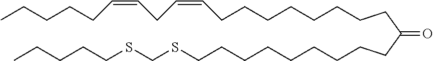 [SMILES: CCCCC/C=C\C/C=C\CCCCCCCCC(=O)CCCCCCCCCSCSCCCCC]

Compound 32 was converted into ketone 18 in 69% yield by the procedure described above for the synthesis of ketone 10. 1H NMR (400 MHZ, CDCl₃) δ5.43-5.28 (m, 4H), 3.67 (s, 2H), 2.77 (t, J=6.5 Hz, 2H), 2.63 (br t, 4H), 2.35 (br t, 4H), 2.17-1.80 (m, 8H), 1.60-1.21 (m, 34H), 0.90 (br t, 6H).

(d) Reduction of ketone: (6Z,9Z,28Z)-heptatriaconta-6,9,28-trien-19-ol (36).

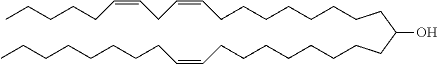 [SMILES: CCCCC/C=C\C/C=C\CCCCCCCCC(O)CCCCCCCC/C=C\CCCCCCCC]

Ketone 16 was converted into alcohol 36 in 95% yield by the procedure described above for the synthesis of alcohol 9. ¹H NMR (400 MHZ, CDCl₃) δ5.34 (m, 6H), 3.57 (m, 1H), 2.77 (t, J=6.2 Hz, 2H), 2.05 (m, 8H), 1.56-1.15 (m, 46H), 0.99 (br t, J=6.2 Hz, 6H).

(e) (6Z,9Z,28Z)-heptatriaconta-6,9,28-trien-19-yl 4-(dimethylamino)butanoate

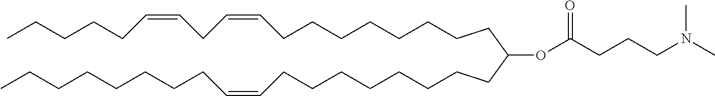 [SMILES: CCCCC/C=C\C/C=C\CCCCCCCCC(CCCCCCCC/C=C\CCCCCCCC)OC(=O)CCCN(C)C]

(33). alcohol 36 was converted into lipid 33 in 79% yield by the procedure described above for the synthesis of lipid 2. ¹H NMR (400 MHZ, CDCl₃) δ5.33 (m, 6H), 4.85 (m, 1H), 2.75 (t, 2H), 2.40 (m, 2H), 2.34 (t, 2H), 2.20 (s, 6H), 2.02 (m, 8H), 1.77 (m, 2H), 1.50 (m, 6H), 1.39-1.17 (m, 40H), 0.87 (br t, 6H).

(f) Representative procedure for reductive amination of ketone: (26Z,29Z)-N-(4-((tert-butyldiphenylsilyl)oxy)butyl)pentatriaconta-26,29-dien-17-amine (38). A

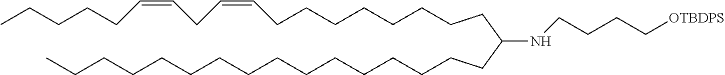

solution of 17 (325 mg, 0.65 mmol), 4-[tert-butyl(diphenyl) silyl]oxybutan-1-amine (318 mg, 0.97 mmol), sodium triacetoxyborohydride (247 mg, 1.17 mmol) and AcOH (1 drop) in 1,2-dichloroethane (4 mL) was stirred at rt under nitrogen for 18 hours. The reaction was quenched with sat. aq. NaHCO₃ (2 mL), diluted with water (4.00 mL) and extracted with CH₂Cl₂ (3×5 mL). The combined extracts were dried (Na₂SO₄) and concentrated. The residue was purified by silica chromatography (0-5% MeOH in DCM) to yield 38 (428 mg, 81%). ¹H NMR (400 MHz, CDCl₃) δ7.70-7.63 (m, 4H), 7.46-7.33 (m, 6H), 5.32 (m, 4H), 3.66 (t, 2H), 2.74 (t, 2H), 2.69-2.41 (m, 3H), 2.02 (m, 4H), 1.82-1.20 (m, 54H), 1.04 (s, 9H), 0.86 (br t, 6H). The following compound was prepared by the same method:

(26Z,29Z)-N-methylpentatriaconta-26,29-dien-17-amine (40). From 17 and

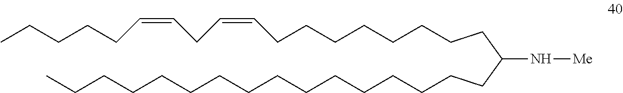 [SMILES: CCCCC/C=C\C/C=C\CCCCCCCCC(CCCCCCCCCCCCCCCC)NC]

MeNH₂ in THF solution. ¹H NMR (400 MHZ, CDCl₃) δ5.32 (m, 4H), 2.74 (t, 2H), 2.55 (m, 1H), 2.12 (s, 3H), 2.02 (m, 4H), 1.82-1.20 (m, 50H), 0.86 (br t, 6H).

(g) Representative procedure for reductive methylation of a secondary amine: (26Z,29Z)-N-(4-((tert-butyldiphenylsilyl)oxy)butyl)-N-methylpentatriaconta-26,29-dien-17-amine (39). A solution of 38 (219 mg, 0.27 mmol), sodium

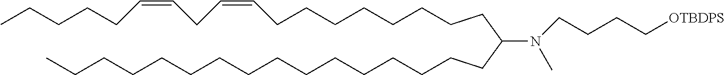

triacetoxyborohydride (284 mg, 1.34 mmol) and aq. formaldehyde (37%, 0.750 mL) in THF (2 mL) was stirred under nitrogen for 2 days. The reaction was quenched with sat. aq. NHCO₃ (2.00 mL), diluted with water (5 mL) and extracted with CH₂Cl₂ (3×5 mL). The combined organics were dried (Na₂SO₄) and concentrated. The residue was purified by silica chromatography (0-5% MeOH in DCM) to yield 39 (174 mg, 78%). ¹H NMR (400 MHz, CDCl₃) δ7.70-7.63 (m, 4H), 7.48-7.33 (m, 6H), 5.32 (m, 4H), 3.66 (t, 2H), 2.74 (t, 2H), 2.67-2.40 (m, 3H), 2.12 (s, 3H), 2.02 (m, 4H), 1.82-1.20 (m, 54H), 1.04 (s, 9H), 0.86 (br t, 6H).

(h) Representative procedure for desilylation: 4-(methyl((26Z,29Z)-pentatriaconta-26,29-dien-17-yl)amino)butan-1-ol (34). To a cold (0° C.) solution of 39

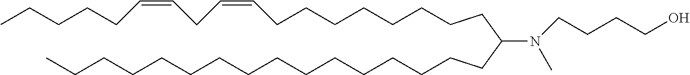 [SMILES: CCCCC/C=C\C/C=C\CCCCCCCCC(CCCCCCCCCCCCCCCC)N(C)CCCCO]

(149 mg, 0.18 mmol) in in THF (2 mL) maintained under nitrogen atmosphere was added HF-pyridine (0.2 mL). The mixture was warmed to room temperature and stirred for 18 hours, then it was diluted with water (5 mL) and extracted with CH₂Cl₂ (3×5 mL). The combined extracts were dried (Na₂SO₄) and concentrated. The residue was purified by silica chromatography (0-5% MeOH in DCM) to yield 34 (76 mg, 71%). ¹H NMR (400 MHZ, CDCl₃) δ5.32 (m, 4H), 3.68 (t, 2H), 2.74 (t, 2H), 2.67-2.40 (m, 3H), 2.12 (s, 3H), 2.02 (m, 4H), 1.82-1.20 (m, 54H), 0.86 (br t, 6H).

(i) Representative procedure for the S_{N}2 hydroxyalkylation of a secondary amine: 4-(methyl((26Z,29Z)-pentatriaconta-26,29-dien-17-yl)amino)butan-1-ol (34). A solution of 40 (260 mg, 0.5 mmol, 1 equiv) and 4-bromo-1-butanol (92 mg, 0.6 mmol, 1.2 equiv) in acctonitrile (1 mL) containing suspended, powdered K₂CO₃ (143 mg, 1 mmol, 2 equiv) in a thick-walled glass tube sealed with a Teflon screw cap was heated to 80° C. for 48 h. The mixture was then cooled, diluted with water (4 mL), and extracted with 1:1 ether-hexane (3×10 mL). The combined extracts were dried (Na₂SO₄) and concentrated and the residue of crude 34 was purified as described in part (h) to give 198 mg (68%) of pure product. NMR data are as in part (h) above.

(j) Representative procedure for ketalization of ketone: 2-(2-((9Z,12Z)-octadeca-9,12-dien-1-yl)-2-(9-(((pentylthio)methyl)thio)nonyl)-1,3-dioxolan-4-yl)ethan-1-ol

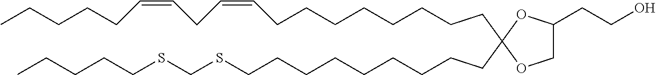 [SMILES: CCCCC/C=C\C/C=C\CCCCCCCCC1(CCCCCCCCCSCSCCCCC)OCC(CCO)O1]

A solution of ketone 18 (550 mg, 1.0 mmol), 1,2,4-butanetriol (technical grade, ca. 90%, 236 mg, 2 mmol) and pyridinium p-toluenesulfonate (50 mg, 0.2 mmol) in 50 mL of toluene was refluxed under nitrogen overnight with continuous removal of water (Dean-Stark trap). Upon completion of the reaction, the mixture was cooled to room temperature, washed with water (2×30 mL), dried by passing over a plug of anhydrous Na₂SO₄ and evaporated. The yellowish oily residue (0.6 g) was purified by silica gel column chromatography with dichloromethane as the eluent, to afford 580 mg (0.9 mmol, 9%) of pure ketal. ¹H NMR (400 MHZ, CDCl₃) δ5.43-5.28 (m, 4H), 4.24 (m, 1H), 4.09 (m, 1H), 3.81 (m, 2H), 3.67 (s, 2H), 3.54 (t, 1H), 2.78 (t, J=6.5 Hz, 2H), 2.63 (br t, 4H), 2.17-1.80 (m, 8H), 1.60-1.21 (m, 40H), 0.90 (br t, 6H).

(j) Representative procedure for conversion of ketal to a lipid: N,N-dimethyl-2-(2-((9Z,12Z)-octadeca-9,12-dien-1-yl)-2-(9-(((pentylthio)methyl)thio)nonyl)-1,3-dioxolan-4-yl)ethan-1-amine (35). Neat methanesulfonyl anhydride (290 mg, 1.6 mmol,

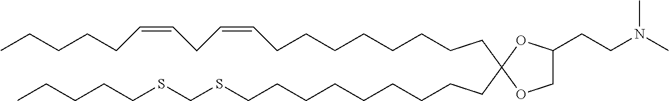 [SMILES: CCCCC/C=C\C/C=C\CCCCCCCCC1(CCCCCCCCCSCSCCCCC)OCC(CCN(C)C)O1]

2 equiv.) was added to a solution of ketal 41 (512 mg, 0.8 mmol, 1 equiv) and dry triethylamine (242 mg, 330 uL, 2.4 mmol) in 5 mL of dry CH₂Cl₂. The resulting mixture was stirred at room temperature overnight. The mixture was diluted with 25 mL of CH₂Cl2. the organic phase was washed with water (2×30 mL), passed over a plug of anhydrous Na₂SO₄, and evaporated to afford 598 mg of crude mesylate as yellowish oil. Without purification, the crude mesylate was added to 20 mL of a commercial 2.0 M solution of dimethylamine in THF. The resulting mixture was stirred at room temperature for 6 days, whereupon no more mesylate was apparent by TLC and/or ¹H NMR. Evaporation of the volatiles returned an oily residue that was purified by column chromatography on silica gel with 0-5% gradient methanol in dichloromethane, resulting in recovery of 467 mg (87%) of pure 35. 1H NMR (400 MHZ, CDCl₃) δ5.43-5.28 (m, 4H), 4.07 (m, 2H), 3.67 (s, 2H), 3.49 (t, 1H), 2.78 (t, J=6.5 Hz, 2H), 2.63 (br t, 4H), 2.23 (s, 6H), 2.20-1.80 (m, 10H), 1.60-1.21 (m, 40H), 0.90 (br t, 6H).

### Example 3: Production of Symmetrical and Unsymmetrical Ketone Building Blocks for Ionizable Lipids by Alkylation of the Ogura Reagent

(a) Representative procedure for the dialkylation of the Ogura reagent:

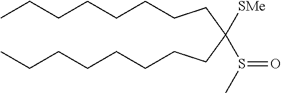 [SMILES: CCCCCCCCC(CCCCCCCC)(SC)S(C)=O]

methyl (9-(methylsulfinyl)heptadecan-9-yl)sulfane (51). Ogura reagent (610 mg, 0.5 mL, 4.92 mmol, 1.0 equiv) was added dropwise over 10 min (syringe pump) to a vigorously stirred suspension of potassium hydride (35% wt in oil, 1.69 g, 14.76 mmol, 3.0 equiv.) in dry THF (4 mL) at 0° C. and under nitrogen. The resulting mixture was stirred for 10 min at 0° C. prior to the addition of 1-bromooctane (1.9 mL, 10.82 mmol, 2.2 equiv). The mixture was allowed to warm to room temperature and stirred at rt for 18 h, then it was cooled 0° C. and carefully quenched with water (25 mL). The mixture was extracted with diethyl ether (3×25 mL) and the combined extracts were washed with brine, dried (Na₂SO₄), filtered, and concentrated in vacuo. The residue was purified by flash column chromatography (95:5 CH₂Cl₂-acetone) to afford 51 (886 mg, 2.54 mmol, 52%) as a colorless oil. ¹H NMR (300 MHz, CDCl₃): δ2.65 (s, 3H) 3H, 2.25 (s, 3H), 1.84-1.13 (m, 28H), 0.9 (br t, 6H).

(b) Representative procedure for the monoalkylation of the Ogura reagent:

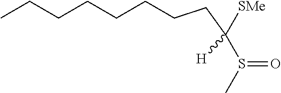

methyl (1-methylsulfinyl)nonyl)sulfane (53). Ogura reagent (2 g, 16.1 mmol, 2.0 equiv) was added dropwise over 15 min (syringe pump) to a vigorously stirred suspension of sodium hydride (60% wt in oil, 645 mg, 16.1 mmol, 2.0 equiv) in dry THF (10 mL) at room temperature and under nitrogen. The resulting mixture was stirred for 30 min at room temperature, then 1-bromooctane (1.4 mL, 8.05 mmol, 1 equiv) was added quickly. The mixture was warmed to 50° C. and stirred at that temperature for 6 hr, then it was carefully poured into a cold (0° C.) saturated aqueous solution of ammonium chloride and extracted with diethyl ether (3×25 mL). The combined extracts were washed with brine, dried (Na₂SO₄), filtered, and concentrated in vacuo. The residue was purified by flash column chromatography eluting with dichloromethane:acetone (95:5) to afford 53 (1.182 g, 5.34 mmol, 66%), colorless oil, as a mixture of diastercomers. ¹H NMR (300 MHz, CDCl₃): δ3.50 (dd, J=3.6, 1.8 Hz) & 3.47 (dd, J=3.6, 1.4 Hz) 1H, 2.73 (s) & 2.57 (s) 3H, 2.30 (s) & 2.18 (s) 3H, 1.83-1.13 (m, 14 H), 0.9 (t, 3H, J=6.5 Hz).

(c) Representative procedure for the alkylation of monoalkyl derivative of the Ogura reagent: methyl(6-(methylsulfinyl)tetradecan-6-yl)sulfane (54). A solution

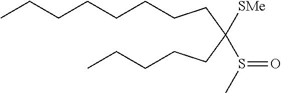 [SMILES: CCCCCCCCC(CCCCC)(SC)S(C)=O]

of methyl(1-(methylsulfinyl)nonyl)sulfane (236 mg, 1 mmol) in dry THF (1 mL) was added dropwise over 10 min (syringe pump) to a vigorously stirred suspension of potassium hydride (35% wt in oil, 170 mg, 1.5 mmol, 1.5 equiv.) in dry THF (1 mL), at 0° C. and under nitrogen. The resulting mixture was stirred for 10 min at 0° C. prior to the addition of 1-bromopentane (168 mg, 1.1 mmol, 1.1 equiv). The mixture was allowed to warm to room temperature and stirred at rt for 18 h, then it was cooled 0° C. and carefully quenched with water (20 mL). The mixture was extracted with diethyl ether (3×15 mL) and the combined extracts were washed with brine, dried (Na₂SO₄), filtered, and concentrated in vacuo. The residue was purified by flash column chromatography (95:5 CH₂Cl₂-acetone) to afford 54 (155 mg, 0.51 mmol, 51%) as a colorless oil. ¹H NMR (300 MHZ, CDCl₃): δ2.65 (s, 3H) 3H, 2.25 (s, 3H), 1.84-1.13 (m, 24H), 0.9 (br t, 6H).

(b) Representative procedure for the hydrolysis of a dialkylated derivative of the Ogura reagent to a ketone: 9-heptadecanone (52). Aqueous 1 M HCl solution (1

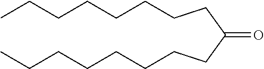 [SMILES: CCCCCCCCC(=O)CCCCCCCC]

mL) was added to a solution of methyl (9-(methylsulfinyl)heptadecan-9-yl)sulfane (35 mg, 0.1 mmol) in ethanol (1 mL) at room temperature, and the mixture was heated to 60° C. and stirred at that temperature for 8 h. The solution was cooled and concentrated in vacuo. The residue was dissolved in CH₂Cl₂ (25 mL) and the solution was washed with brine, dried (Na₂SO₄), filtered, and concentrated in vacuo to afford 52 (25 mg, 0.1 mmol, ˜quant) as an off-white solid, m.p. 48-49° C., lit. m.p. 50-51° C. ¹H NMR (300 MHz, CDCl₃): δ2.36 (t, 4H, J=7.4 Hz), 1.60-1.20 (m, 24 H), 0.85 (br t, 6H). ¹³C NMR (75 MHZ, CDCl₃): δ211.8, 42.8, 31.8, 29.4, 29.3, 29.2, 23.9, 22.7, 14.1. The following ketone was prepared by the same method:

6-tetradecanone (55). From methyl(6-(methylsulfinyl)tetradecan-6-yl)sulfane.

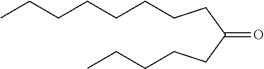

Low-melting semisolid (lit. m.p. 25° C.). ¹H NMR (300 MHz, CDCl₃): δ2.38 (br t, 4H), 1.59-1.20 (m, 18 H), 0.88 (br t, 6H).

The foregoing examples are illustrative only. That is, various alterations can be made without departing from the scope of certain aspects of the invention as described herein.

1. A method for producing an ionizable, cationic amino lipid, the method comprising:
(i) reacting a double alkylated nucleophilic intermediate under conditions effective to convert the intermediate to a ketone,
wherein the double alkylated nucleophilic intermediate has a structure as defined below: 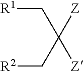
wherein Z is isocyano and Z′ is tosyl, or wherein Z is SCH₃ and Z′ is S(O)CH₃;
wherein the ketone has a structure as defined below: 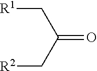
wherein the R¹ and R² groups are linear, branched and/or cyclic, optionally substituted C₃ to C₃₀ alkyl groups, optionally comprising 0-3 carbon-carbon double bonds, optionally comprising heteroatoms selected from N, O and/or S, optionally comprising homocyclic or heterocyclic ring structures; and
wherein R¹ and R² are identical or different; and
(ii) preparing the ionizable, cationic amino lipid from the ketone thereof using one or more synthesis steps resulting in an addition of an ionizable head group moiety to (a) the ketone; or (b) an alcohol produced from an optional reduction of the ketone to produce the alcohol, thereby producing the ionizable, cationic amino lipid, wherein the ionizable, cationic amino lipid has a protonatable amino head group and comprises two lipophilic chains comprising the R¹ and R² groups respectively and wherein the ionizable, cationic amino lipid has (i) a pKa of between 6 and 7.5; and (ii) a logP of at least 11.

2. The method of claim 1, wherein the conditions effective to convert the double alkylated nucleophilic intermediate to the ketone comprise addition of a mineral acid.

3. The method of claim 2, wherein the mineral acid is HCl, H₂SO₃, H₃PO₄ or a combination thereof.

4. The method of claim 1, wherein the double alkylated intermediate is produced by alkylating a reagent A selected from 1-((isocyanomethyl)sulfonyl)-4-methylbenzene (tosylmethyl isocyanide; TosMIC) or methyl((methylsulfinyl)methyl) sulfane (Ogura reagent) with at least one alkyl halide or sulfonate.

5. The method of claim 4, wherein R¹ and R² are identical, such that the ionizable, cationic amino lipid so produced is symmetrical, and wherein the double alkylated nucleophilic intermediate is produced upon treatment of the reagent A with a base and the alkyl halide or sulfonate.

6. The method of claim 5, wherein the double alkylated nucleophilic intermediate is produced using two or more molar equivalents each of the base and the alkyl halide or sulfonate.

7. The method of claim 4, wherein R¹ and R² are different, such that the ionizable, cationic amino lipid so produced is unsymmetrical, and wherein the ketone is produced by a reaction scheme as defined below comprising treating the reagent A with a first base and a first of the at least one alkyl halide or sulfonate having structure B to produce an alkylated intermediate of structure E, and reacting the structure E with a second base that is the same or different than the first base with a second of the at least one alkyl halide or sulfonate having structure F to produce the double alkylated nucleophilic intermediate G and wherein the conditions effective to produce the ketone H comprise reacting the double alkylated nucleophilic intermediate G with an aqueous solution of a strong mineral acid,

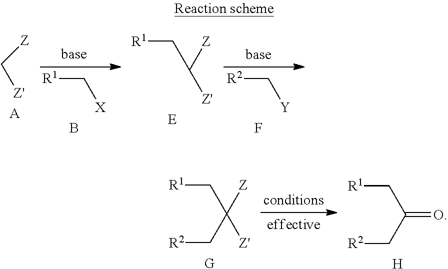

8. The method of claim 7, wherein synthetic intermediate E is reacted with about one or more molar equivalent each of the second base and the structure F alkyl halide or sulfonate.

9. The method of claim 7, wherein the reagent A is methyl((methylsulfinyl)methyl)sulfane (Ogura reagent, Ogura sulfoxide) and the first and/or second base is potassium hydride.

10. The method of claim 7, wherein the reagent A is 1-((isocyanomethyl)sulfonyl)-4-methylbenzene (tosyl methyl isonitrile, TosMIC) and the first and/or second base is sodium hydride.

11. The method of claim 1, wherein the ketone is converted into the corresponding alcohol by the reduction reaction comprising addition of the sodium borohydride in the solvent.

12. The method of claim 1, wherein the ketone is converted into the ionizable, cationic amino lipid by a ketalization with an aminodiol hydrochloride.

13. The method of claim 7, wherein X and Y are selected from Cl, Br, I, tosyloxy and mesyloxy.

14. The method of producing the ionizable, cationic amino lipid of claim 1, wherein the ketone is reacted, using one or more synthetic steps, to produce the ionizable, cationic amino lipid and wherein the ionizable, cationic amino lipid has a structure as defined by structure K: 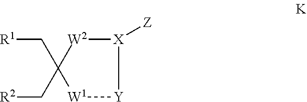
or a pharmaceutically acceptable salt thereof,
wherein:
W¹ and Y are either bonded to each other or not bonded to each other (as indicated by the dashed bond), and:
if W¹ and Y are bonded to each other then:
W¹ is O or S,
W² is O or S,
X is CH,
Y is (CH₂)ₘ, wherein m is 1 or 2;
Z is a group selected from structures a-c below: 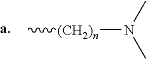
type 2 ionizable head; 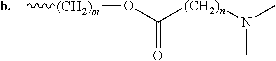
type 3 ionizable head; and 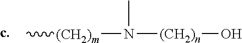
type 4 ionizable head,
wherein the wavy line represents the bond to X,
if W¹ and Y are not bonded to each other, then:
W¹ is H;
W² is O or S or NH or NR², wherein R² is a C₁ to C₄ small alkyl optionally substituted with an OH group;
Group 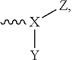
wherein the wavy line represents the bond to W², is a group chosen from among structures d-I below: 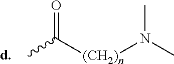
if W² is O, type 1 ionizable head; 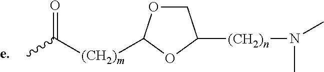
if W² is O, type 5 ionizable head; 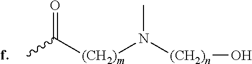
if W² is O, type 6 ionizable head; 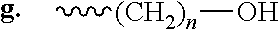
it W² is NH or NR², type 7 ionizable head; 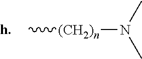
if W² is NH or NR², type 8 ionizable head; and 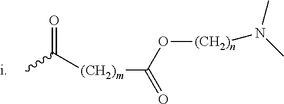
if W² is O, type 9 ionizable head, wherein the wavy line represents the bond to W².

15. The method of claim 14, wherein the ionizable, cationic amino lipid is MC3 or KC2.

16. The method of claim 1, further comprising formulating the ionizable, cationic amino lipid within a lipid nanoparticle.

17. The method of claim 16, wherein the ionizable, cationic amino lipid is co-formulated with nucleic acid, non-cationic lipid and optionally a conjugated lipid that inhibits aggregation of particles.

18. The method of claim 17, wherein the non-cationic lipid is phosphatidylcholine, cholesterol or a combination thereof and wherein the conjugated lipid is a hydrophilic polymer-lipid conjugate.

19. Use of a double alkylated nucleophilic intermediate to produce an ionizable, cationic amino lipid, the double alkylated nucleophilic intermediate having a structure as defined below: 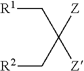
wherein Z is SCH₃ and Z′ is S(O)CH₃, or wherein Z is isocyano and Z′ is tosyl;
wherein the R¹ and R² groups are linear, branched and/or cyclic, optionally substituted C₃ to C₃₀ alkyl groups, optionally comprising 0-3 carbon-carbon double bonds, optionally comprising one or more heteroatoms selected from N, O and/or S, optionally comprising one or more homocyclic or heterocyclic ring structures; and
wherein R¹ and R² are identical or different.
